SEQUENCE LISTING (28 sequences)

SEQ ID NO: 1 - protein, 7 aa, Artificial sequence
  synthetic
  X (1)..(1): Xaa is aspartic acid, glutamic acid, alanine, or glycosylated forms thereof
  X (2)..(2): Xaa is arginine, histidine, lysine, or glycosylated forms thereof
  X (3)..(3): Xaa is valine, alanine, isoleucine, leucine, or glycosylated forms thereof
  X (4)..(4): Xaa is tyrosine, phenylalanine, tryptophan, or glycosylated forms thereof
  X (5)..(5): Xaa is isoleucine, valine, alanine, leucine, or glycosylated forms thereof
  X (6)..(6): Xaa is histidine, arginine, lysine, or glycosylated forms thereof
  X (7)..(7): Xaa is proline, glycine, serine, or glycosylated forms thereof
XXXXXXX

SEQ ID NO: 2 - protein, 7 aa, Artificial sequence
  synthetic
DRVYIHP

SEQ ID NO: 3 - protein, 7 aa, Artificial sequence
  synthetic
  P (7)..(7): glycosylated
DRVYIHP

SEQ ID NO: 4 - protein, 7 aa, Artificial sequence
  synthetic
DRVYIHP

SEQ ID NO: 5 - protein, 7 aa, Artificial sequence
  synthetic
  P (7)..(7): glycosylated
DRVYIHP

SEQ ID NO: 6 - protein, 8 aa, Artificial sequence
  synthetic
DRVYIHPS

SEQ ID NO: 7 - protein, 8 aa, Artificial sequence
  synthetic
  S (8)..(8): glycosylated
DRVYIHPS

SEQ ID NO: 8 - protein, 8 aa, Artificial sequence
  synthetic
DRVYIHPS

SEQ ID NO: 9 - protein, 8 aa, Artificial sequence
  synthetic
  S (8)..(8): glycosylated
DRVYIHPS

SEQ ID NO: 10 - protein, 7 aa, Artificial sequence
  synthetic
DRVYIHS

SEQ ID NO: 11 - protein, 7 aa, Artificial sequence
  synthetic
  S (7)..(7): glycosylated
DRVYIHS

SEQ ID NO: 12 - protein, 7 aa, Artificial sequence
  synthetic
DRVYIHS

SEQ ID NO: 13 - protein, 7 aa, Artificial sequence
  synthetic
  S (7)..(7): glycosylated
DRVYIHS

SEQ ID NO: 14 - protein, 7 aa, Artificial sequence
  synthetic
ARVYIHP

SEQ ID NO: 15 - protein, 7 aa, Artificial sequence
  synthetic
  P (7)..(7): glycosylated
ARVYIHP

SEQ ID NO: 16 - protein, 7 aa, Artificial sequence
  synthetic
ARVYIHP

SEQ ID NO: 17 - protein, 7 aa, Artificial sequence
  synthetic
  P (7)..(7): glycosylated
ARVYIHP

SEQ ID NO: 18 - protein, 8 aa, Artificial sequence
  synthetic
ARVYIHPS

SEQ ID NO: 19 - protein, 8 aa, Artificial sequence
  synthetic
  S (8)..(8): glycosylated
ARVYIHPS

SEQ ID NO: 20 - protein, 8 aa, Artificial sequence
  synthetic
ARVYIHPS

SEQ ID NO: 21 - protein, 8 aa, Artificial sequence
  synthetic
  S (8)..(8): glycosylated
ARVYIHPS

SEQ ID NO: 22 - protein, 7 aa, Artificial sequence
  synthetic
ARVYIHS

SEQ ID NO: 23 - protein, 7 aa, Artificial sequence
  synthetic
  S (7)..(7): glycosylated
ARVYIHS

SEQ ID NO: 24 - protein, 7 aa, Artificial sequence
  synthetic
ARVYIHS

SEQ ID NO: 25 - protein, 7 aa, Artificial sequence
  synthetic
  S (7)..(7): glycosylated
ARVYIHS

SEQ ID NO: 26 - protein, 7 aa, Artificial sequence
  synthetic
  X (1)..(1): Xaa is Asp, Glu, Ala, Asn, Pro, Gly, or glycosylated forms thereof
  X (2)..(2): Xaa is Arg, Lys, Ala, or glycosylated forms thereof
  X (3)..(3): Xaa is Nle, Val, Leu, Ile, Ala, Gly, Lys, Pro, Tyr, or glycosylated forms thereof
  X (4)..(4): Xaa is Tyr, Ser, Thr, Ala, or glycosylated forms thereof
  X (5)..(5): Xaa is Ile, Val, Leu, Nle, Ala, Gly, or glycosylated forms thereof
  X (6)..(6): Xaa is His, Arg, or glycosylated forms thereof
  X (7)..(7): Xaa is Pro, Ser, Cys, or glycosylated forms thereof
XXXXXXX

SEQ ID NO: 27 - protein, 8 aa, Artificial sequence
  synthetic
  X (1)..(1): Xaa is Asp, Glu, Ala, Asn, Pro, Gly, or glycosylated forms thereof
  X (2)..(2): Xaa is Arg, Lys, Ala, or glycosylated forms thereof
  X (3)..(3): Xaa is Nle, Val, Leu, Ile, Ala, Gly, Lys, Pro, Tyr, or glycosylated forms thereof
  X (4)..(4): Xaa is Tyr, Ser, Thr, Ala or glycosylated forms thereof
  X (5)..(5): Xaa is Ile, Val, Leu, Nle, Ala, Gly
  X (6)..(6): Xaa is His, Arg, or glycosylated forms thereof
  X (7)..(7): Xaa is Pro, Ala
  X (8)..(8): Xaa is Phe, Ser, Cys, Ile, Tyr, or glycosylated forms thereof
XXXXXXXX

SEQ ID NO: 28 - protein, 8 aa, Artificial sequence
  synthetic
  X (1)..(1): Xaa is aspartic acid, glutamic acid, alanine, or glycosylated forms thereof
  X (2)..(2): Xaa is arginine, histidine, lysine, or glycosylated forms thereof
  X (3)..(3): Xaa is valine, alanine, isoleucine, leucine, or glycosylated forms thereof
  X (4)..(4): Xaa is tyrosine, phenylalanine, tryptophan, or glycosylated forms thereof
  X (5)..(5): Xaa is isoleucine, valine, alanine, leucine, or glycosylated forms thereof
  X (6)..(6): Xaa is histidine, arginine, lysine, or glycosylated forms thereof
  X (7)..(7): Xaa is proline, glycine, serine, or glycosylated forms thereof
  X (8)..(8): Xaa is serine, threonine, hydroxyproline, or glycosylated forms thereof
XXXXXXXX

(19) United States
(12) Patent Application Publication
(10) Pub. No.: US 2024/0012012 A1
(43) Pub. Date: Jan. 11, 2024

(54) BIOMARKER AND TREATMENT FOR VASCULAR COGNITIVE IMPAIRMENT AND RELATED CONDITIONS

(71) Applicants: Meredith HAY, Tucson, AZ (US); John KONHILAS, Tucson, AZ (US); Christina HOYER-KIMURA, Tucson, AZ (US); Thomas BEACH, Sun City, AZ (US); Geidy SERRANO, Sun City, AZ (US); Eric REIMAN, Phoenix, AZ (US)

(72) Inventors: Meredith HAY, Tucson, AZ (US); John KONHILAS, Tucson, AZ (US); Christina HOYER-KIMURA, Tucson, AZ (US); Thomas BEACH, Sun City, AZ (US); Geidy SERRANO, Sun City, AZ (US); Eric REIMAN, Phoenix, AZ (US)

(21) Appl. No.: 18/039,641

(22) PCT Filed: Dec. 1, 2021
(86) PCT No.: PCT/US2021/061394

§ 371 Date: May 31, 2023

Related U.S. Application Data
(60) Provisional application No. 63/119,917, filed on Dec. 1, 2020.

(51) Int. Cl. G01N 33/68; C07K 14/515; A61P 25/28

(52) U.S. Cl. CPC G01N 33/6896; C07K 14/515; A61P 25/28; G01N 2800/52

(57) ABSTRACT

The present invention provides compositions and methods useful in the diagnosis and treatment of a variety of cognitive impairments including vascular cognitive impairment and dementia, and Alzheimer's Disease. In one particular embodiment, the level of the neurofilament light protein and/or phosphorylated tau (e.g., p-tau181) is used to diagnose a cognitive impairment or monitor the efficacy of a therapy.

### CROSS-REFERENCE TO RELATED APPLICATION

The present application claims priority to U.S. Provisional Patent Application No. 63/119,917, filed Dec. 1, 2020, which is hereby incorporated by reference in its entirety.

### STATEMENT OF GOVERNMENT SPONSOR RESEARCH

This invention was made with government support under Grant No. U01 AG066623, awarded by National Institutes of Health. The government has certain rights in the invention.

### FIELD OF THE INVENTION

The present invention relates to compositions and methods useful in the diagnosis and treatment of a variety of cognitive impairments including vascular cognitive impairment and dementia, and Alzheimer's Disease.

### BACKGROUND OF THE INVENTION

Vascular cognitive impairment (VCI), also known as vascular dementia, multi-infarct dementia (MID), and vascular contributions to cognitive impairment and dementia (VCID) are caused by impairments in blood supply to the brain. These conditions and terms refer to a syndrome consisting of a complex interaction of cerebrovascular disease and risk factors leading to changes in the brain structures (strokes, lesions), and resulting changes in cognition. Both VCI and Alzheimer's disease-related dementias (ADRDs) are strongly correlated with increases in systemic and brain inflammatory cytokines, increased reactive oxygen species (ROS) and/or reduced brain blood flow. Each of these conditions individually and in combination contribute to and accelerate the progression of VCI and ADRD.

There exists a need to provide simplified methodology for diagnosing VCI and Alzheimer's disease (AD), as well as treating and monitoring the efficacy of treatment in subjects suspected of having or diagnosed as having VCI or AD.

### SUMMARY OF THE INVENTION

Experiments conducted during the course of developing embodiments for the present invention demonstrated that 1) blood-based measurements of serum neurofilament light protein (NFL) is increased in a murine model of VCID as well as in patients with diagnosed VCID, and 2) treatment with Ang-(1-7) derivative oligopeptides (e.g., PN-A5) modifies NFL and is correlated with levels of brain inflammatory cytokines and cognitive dysfunction.

Indeed, additional experiments demonstrated that treatment with Ang-(1-7)/MasR agonists reversed VCID-induced cognitive impairment and significantly decreased NfL levels in a mouse model of VCID as compared to HF-saline treated mice. Further, NfL levels were shown to be significantly negatively correlated with cognitive scores and the concentrations of multiple pleiotropic cytokines in the brain. Such results indicate that treatment with Ang-(1-7)/MasR agonists rescues cognitive impairment and decreases plasma NfL relative to HF-saline-treated animals in a VCID mouse model.

Additional experiments demonstrated increases in circulating neurodegeneration biomarkers neurofilament light protein and phosphorylated tau181 (e.g., p-tau181) in individuals with preserved cardiac function heart failure. Moreover, such experiments further demonstrated that serum NfL and pTau increased with severity of heart failure.

Accordingly, the present invention provides compositions and methods useful in the diagnosis and treatment of a variety of cognitive impairments including vascular cognitive impairment and dementia, and Alzheimer's Disease. In one particular embodiment, the level of the neurofilament light protein and/or phosphorylated tau (e.g., p-tau181) is used to diagnose a cognitive impairment or monitor the efficacy of a therapy.

In one aspect, the invention provides a method for treating a subject diagnosed as having cognitive impairment comprising:
(a) determining from a blood fraction a first measurement of the neurofilament light protein (NFL) and/or p-tau (e.g., p-tau181) concentration in the subject;
(b) administering to the subject a therapeutic agent intended to reduce or eliminate at least one symptom of the cognitive impairment;
(c) determining from a blood fraction a second measurement of the serum neurofilament light protein (NFL) and/or p-tau (e.g., p-tau181) concentration in the subject; and
(d) comparing the second measurement to the first measurement and either (i) increasing the dosage, the administration frequency, or both of the therapeutic agent if the second measurement is the greater than or equal to the first measurement or (ii) maintaining or reducing the dosage, the administration frequency, or both of the therapeutic agent if the second measurement is the less than the first measurement.

In another aspect, the invention provides a method for treating a subject diagnosed as having cognitive impairment comprising:
(a) administering to the subject a therapeutic agent intended to reduce or eliminate at least one symptom of the cognitive impairment;
(b) determining from a blood fraction a neurofilament light protein (NFL) and/or p-tau (e.g., p-tau181) concentration in the subject;
(c) comparing the NFL level measured in step (b) to a pre-determined reference level; and
(d) either (i) increasing the dosage, the administration frequency, or both of the therapeutic agent if the NFL level measured in step (b) and/or the p-tau (e.g., p-tau181) level measured in step (b) is the greater than or equal to the reference level or (ii) maintaining or reducing the dosage, the administration frequency, or both of the therapeutic agent if the NFL level measured in step (b) and/or the p-tau (e.g., p-tau181) level measured in step (b) is the less than the reference level.

In some embodiments, symptoms of cognitive impairment include, but are not limited to, progressive loss of memory, cognition, reasoning, judgment, aspects of higher cortical function, diminished initiative, excessive distraction, speech, motor activity, recognition of perceptions, exaggerated or caricatured personality traits, irritability, excessive anger, violence, uncontrollable agitation, and delusions.

In another aspect, the invention provides a method for treating a subject diagnosed as having cognitive impairment comprising:
(a) determining from a blood fraction a neurofilament light protein (NFL) and/or p-tau (e.g., p-tau181) concentration in the subject;
(b) comparing the NFL level measured in step (a) and/or the p-tau (e.g., p-tau181) level measured in step (a) to a pre-determined reference level; and
(c) administering a therapeutically agent to the subject if the NFL level measured in step (a) and/or the p-tau (e.g., p-tau181) level measured in step (a) is greater than the reference level.

In some embodiments, the blood fraction is serum, plasma, or whole blood.

In some embodiments, the cognitive impairment is vascular cognitive impairment, post-surgical cognitive impairment, or Alzheimer's disease.

In some embodiments, the therapeutic agent is a Mas receptor agonist including, for example, a Mas receptor agonist having the amino acid sequence of any one of SEQ ID NOs: 2-27. Optionally, the Mas receptor agonist is glycosylated.

In some embodiments, the Mas receptor agonist is an oligopeptide that is a non-naturally-occurring angiotensin-(1-7) derivative polypeptide, i.e., “Ang-(1-7) derivative.” Oligopeptides of the invention may have a longer in vivo half-life and/or increased blood-brain barrier penetration than Ang-(1-7). In some embodiments, the oligopeptides of the invention have seven or eight amino acids and have biological activity as an agonist of the Mas receptor.

In some embodiments, the Mas receptor agonist is an oligopeptide derivative of the formula: A¹-A²-A³-A⁴-A⁵-A⁶-A⁷-A⁸ (SEQ ID NO:1), where A¹ is selected from the group consisting of aspartic acid, glutamic acid, alanine, and glycosylated forms thereof; A² is selected from the group consisting of arginine, histidine, lysine, and glycosylated forms thereof; A³ is selected from the group consisting of valine, alanine, isoleucine, leucine, and glycosylated forms thereof; A⁴ is selected from the group consisting of tyrosine, phenylalanine, tryptophan, and glycosylated forms thereof; A⁵ is selected from the group consisting of isoleucine, valine, alanine, leucine, and glycosylated forms thereof; A⁶ is selected from the group consisting of histidine, arginine, lysine, and glycosylated forms thereof; A⁷ is selected from the group consisting of proline, glycine, serine, and glycosylated forms thereof; and A⁸ can be present or absent, wherein when A⁸ is present, A⁸ is selected from the group consisting of serine, threonine, hydroxyproline, and glycosylated forms thereof, provided (i) at least one of A¹-A⁸ is optionally substituted with a mono- or di-carbohydrate; or (ii) when A⁸ is absent: (a) at least one of A¹-A⁷ is substituted with a mono- or di-carbohydrate, (b) A⁷ is terminated with an amino group, or (c) a combination thereof.

In some embodiments, carbohydrate comprises glucose, galactose, xylose, fucose, rhamnose, lactose, cellobiose, melibiose, or a combination thereof. In other embodiments, A⁸ is serine or a glycosylated form thereof, or A⁸ is absent and A⁷ is serine or a glycosylated form thereof. In some embodiments, only the C-terminal amino acid is glycosylated (e.g., A⁸ or A⁷ when A⁸ is absent).

Still in other embodiments, (i) A⁸ is terminated with an amino group; or (ii) when A⁸ is absent, A⁷ is terminated with an amino group. Within these embodiments, in some instances (i) A⁸ is serine that is optionally glycosylated (e.g., with glucose or lactose); or (ii) when A⁸ is absent, A⁷ is serine that is optionally glycosylated (e.g., with glucose or lactose). Still in other instances, when A⁸ is absent and A⁷ serine that is glycosylated with glucose. Within the latter instances, in some cases A⁷ is terminated with an amino group. In some embodiments, whether or not the Ang(1-7) derivative is terminated with an amino group, the C-terminal amino acid (A⁸ or A⁷ when A⁸ is absent) is the only glycosylated amino acid.

Yet in other embodiments, A¹ is aspartic acid; A² is arginine; A³ is valine; A⁴ is tyrosine; A⁵ is isoleucine; A⁶ is histidine; and (i) A⁸ is absent and A⁷ is terminated with an amino group or A⁷ is a glycosylated serine, or (ii) A⁸ is serine terminated with an amino group. Within these embodiments, in some cases A⁸ is a glycosylated serine. Still in other cases, A⁸ is absent and A⁷ is a glycosylated serine that is terminated with an amino group.

In some embodiments, the Mas receptor agonist is native Ang(1-7), PN-A5, or PN-A6.

In another aspect, the invention provides a method for diagnosing cognitive impairment in a human subject comprising:
(a) determining from a blood fraction a neurofilament light protein (NFL) concentration and/or p-tau (e.g., p-tau181) concentration in the subject;
(b) comparing the NFL level measured in step (a) and/or the p-tau (e.g., p-tau181) level measured in step (a) to a pre-determined reference level; and
(c) diagnosing the subject as having cognitive impairment when the NLF level measured in step (a) and/or the p-tau (e.g., p-tau181) level measured in step (a) exceed the reference level.

In some embodiments of any of the foregoing aspects, the cognitive impairment is vascular cognitive impairment, post-surgical cognitive impairment, or Alzheimer's disease.

In some embodiments of any of the foregoing aspects, the reference level is about 25, 30, 35, 40, 45, 50, 60, 65, 70, or 75 pg/ml in serum.

In some embodiments of any of the foregoing aspects, the subject is human.

In one aspect, the invention provides a method for treating a subject diagnosed as having cognitive impairment comprising:
(a) determining from a blood fraction a first measurement of the neurofilament light protein (NFL) concentration in the subject;
(b) administering to the subject a therapeutic agent intended to reduce or eliminate at least one symptom of the cognitive impairment;
(c) determining from a blood fraction a second measurement of the serum neurofilament light protein (NFL) concentration in the subject; and
(d) comparing the second measurement to the first measurement and either (i) increasing the dosage, the administration frequency, or both of the therapeutic agent if the second measurement is the greater than or equal to the first measurement or (ii) maintaining or reducing the dosage, the administration frequency, or both of the therapeutic agent if the second measurement is the less than the first measurement.

In another aspect, the invention provides a method for treating a subject diagnosed as having cognitive impairment comprising:
(a) administering to the subject a therapeutic agent intended to reduce or eliminate at least one symptom of the cognitive impairment;
(b) determining from a blood fraction a neurofilament light protein (NFL) concentration in the subject;
(c) comparing the NFL level measured in step (b) to a pre-determined reference level; and
(d) either (i) increasing the dosage, the administration frequency, or both of the therapeutic agent if the NFL level measured in step (b) is the greater than or equal to the reference level or (ii) maintaining or reducing the dosage, the administration frequency, or both of the therapeutic agent if the NFL level measured in step (b) is the less than the reference level.

In some embodiments, symptoms of cognitive impairment include, but are not limited to, progressive loss of memory, cognition, reasoning, judgment, aspects of higher cortical function, diminished initiative, excessive distraction, speech, motor activity, recognition of perceptions, exaggerated or caricatured personality traits, irritability, excessive anger, violence, uncontrollable agitation, and delusions.

In another aspect, the invention provides a method for treating a subject diagnosed as having cognitive impairment comprising:
(a) determining from a blood fraction a neurofilament light protein (NFL) concentration in the subject;
(b) comparing the NFL level measured in step (a) to a pre-determined reference level; and
(c) administering a therapeutically agent to the subject if the NFL level measured in step (a) is greater than the reference level.

In some embodiments, the blood fraction is serum, plasma, or whole blood.

In some embodiments, the cognitive impairment is vascular cognitive impairment, post-surgical cognitive impairment, or Alzheimer's disease.

In some embodiments, the therapeutic agent is a Mas receptor agonist including, for example, a Mas receptor agonist having the amino acid sequence of any one of SEQ ID NOs: 2-27. Optionally, the Mas receptor agonist is glycosylated.

In some embodiments, the Mas receptor agonist is an oligopeptide that is a non-naturally-occurring angiotensin-(1-7) derivative polypeptide, i.e., “Ang-(1-7) derivative.” Oligopeptides of the invention may have a longer in vivo half-life and/or increased blood-brain barrier penetration than Ang-(1-7). In some embodiments, the oligopeptides of the invention have seven or eight amino acids and have biological activity as an agonist of the Mas receptor.

In some embodiments, the Mas receptor agonist is an oligopeptide derivative of the formula: A¹-A²-A³-A⁴-A⁵-A⁶-A⁷-A⁸ (SEQ ID NO:1), where A¹ is selected from the group consisting of aspartic acid, glutamic acid, alanine, and glycosylated forms thereof; A² is selected from the group consisting of arginine, histidine, lysine, and glycosylated forms thereof; A³ is selected from the group consisting of valine, alanine, isoleucine, leucine, and glycosylated forms thereof, A⁴ is selected from the group consisting of tyrosine, phenylalanine, tryptophan, and glycosylated forms thereof; A⁵ is selected from the group consisting of isoleucine, valine, alanine, leucine, and glycosylated forms thereof; A⁶ is selected from the group consisting of histidine, arginine, lysine, and glycosylated forms thereof; A⁷ is selected from the group consisting of proline, glycine, serine, and glycosylated forms thereof; and A⁸ can be present or absent, wherein when A⁸ is present, A⁸ is selected from the group consisting of serine, threonine, hydroxyproline, and glycosylated forms thereof, provided (i) at least one of A¹-A⁸ is optionally substituted with a mono- or di-carbohydrate; or (ii) when A⁸ is absent: (a) at least one of A¹-A⁷ is substituted with a mono- or di-carbohydrate, (b) A⁷ is terminated with an amino group, or (c) a combination thereof.

In some embodiments, carbohydrate comprises glucose, galactose, xylose, fucose, rhamnose, lactose, cellobiose, melibiose, or a combination thereof. In other embodiments, A⁸ is serine or a glycosylated form thereof, or A⁸ is absent and A⁷ is serine or a glycosylated form thereof. In some embodiments, only the C-terminal amino acid is glycosylated (e.g., A⁸ or A⁷ when A⁸ is absent).

Still in other embodiments, (i) A⁸ is terminated with an amino group; or (ii) when A⁸ is absent, A⁷ is terminated with an amino group. Within these embodiments, in some instances (i) A⁸ is serine that is optionally glycosylated (e.g., with glucose or lactose); or (ii) when A⁸ is absent, A⁷ is serine that is optionally glycosylated (e.g., with glucose or lactose). Still in other instances, when A⁸ is absent and A⁷ serine that is glycosylated with glucose. Within the latter instances, in some cases A⁷ is terminated with an amino group. In some embodiments, whether or not the Ang(1-7) derivative is terminated with an amino group, the C-terminal amino acid (A⁸ or A⁷ when A⁸ is absent) is the only glycosylated amino acid.

Yet in other embodiments, A¹ is aspartic acid; A² is arginine; A³ is valine; A⁴ is tyrosine; A⁵ is isoleucine; A⁶ is histidine; and (i) A⁸ is absent and A⁷ is terminated with an amino group or A⁷ is a glycosylated serine, or (ii) A⁸ is serine terminated with an amino group. Within these embodiments, in some cases A⁸ is a glycosylated serine. Still in other cases, A⁸ is absent and A⁷ is a glycosylated serine that is terminated with an amino group.

In some embodiments, the Mas receptor agonist is native Ang(1-7), PN-A5, or PN-A6.

In another aspect, the invention provides a method for diagnosing cognitive impairment in a human subject comprising:
(a) determining from a blood fraction a neurofilament light protein (NFL) concentration in the subject;
(b) comparing the NFL level measured in step (a) to a pre-determined reference level; and
(c) diagnosing the subject as having cognitive impairment when the NLF level measured in step (a) exceed the reference level.

In some embodiments of any of the foregoing aspects, the cognitive impairment is vascular cognitive impairment, post-surgical cognitive impairment, or Alzheimer's disease.

In some embodiments of any of the foregoing aspects, the reference level is about 25, 30, 35, 40, 45, 50, 60, 65, 70, or 75 pg/ml in serum.

In some embodiments of any of the foregoing aspects, the subject is human.

### BRIEF DESCRIPTION OF THE DRAWINGS

FIG. 1A is a box plot demonstrating that Mas receptor agonist therapy reverses the VCI measured by the D-ratio in the NOR test.

FIG. 1B is a line graph demonstrating the relationship between Mas receptor agonist dose and improvement in the NOR test (D-ratio) in a VCI model.

FIG. 1C is a bar graph illustrating the serum NFL concentration (pg/ml) in a murine model of heart failure following treatment with native Ang-(1-7) at 50 μg/kg/day or 500 μg/kg/day (“HF A50” and “HF A500,” respectively), PN-A5 at 50 μg/kg/day or 500 μg/kg/day (“HF P50” and “HF P500,” respectively), saline vehicle control (“HF saline”) or sham-operated saline vehicle control (“Control saline”).

FIG. 2 is a line graph showing the correlation between performance in the novel object recognition (NOR) test, as measured by the D Ratio, and serum NFL concentration (pg/ml) in the murine subjects described in FIG. 1.

FIG. 3 is a line graph showing the correlation between the serum NFL concentration (pg/ml) and composite brain cytokine level in the murine subjects described in FIG. 1.

FIG. 4 is a bar graph showing the serum NFL levels (pg/ml) in human subjects before and after coronary artery bypass grafting (CABG) both with and without treatment with native Ang-(1-7) at 200 μg/kg/day.

FIG. 5 is a bar graph showing the serum NFL concentration (pg/ml) in post-mortem human samples from subjects diagnosed as having VCI and controls.

FIG. 6. Experiment timeline. Three-month-old male mice were acclimated for one week before MI or Control surgeries. 5 weeks following recovery, mice were treated with Saline, Ang-(1-7) or PNA5 at 2 concentrations (50 and 500 ug/kg) for 24 days. During the last 3 days of treatment mice underwent Novel Object Recognition testing. Following Novel Object Recognition testing, mice underwent echocardiographs before killing. At killing, the plasma, brain, and heart were collected. MI myocardial infarct, S.C. subcutaneous injection.

FIG. 7A-C. Echocardiograms and histology to confirm myocardial infarctions in the heart failure model. A Simpson Ejection Fraction was calculated using V-mode left ventricle calculations treatment groups: heart failure (HF)-A50 (Ang-(1-7)₅₀ ug/kg), HF-P50 (PNA5 50 ug/kg), HF-A500 (Ang-(1-7) 500 ug/kg), HF-P500 (PNA5 500 ug/kg), HF-Saline, Control-Saline. Significant difference between Control-Saline and the following treatment groups are as listed: HF-Saline, HF-A50 (Ang-(1-7)₅₀ ug/kg), HF-P50 (PNA5 50 ug/kg), HF-A500 (Ang-(1-7)₅₀₀ ug/kg), HF-P500 (PNA5 500 ug/kg), P<0.05 ANOVA, Dunnett's post. B Simpson End Systolic Volume was calculated using V-mode left ventricle calculation. Significant difference between Control-Saline and the following treatment groups are as listed: HF-Saline p=0.001, HF-P50 (PNA5 50 ug/kg) p=0.0001, and HF-A500 (Ang-(1-7)₅₀₀ ug/kg) p=0.015, ANOVA, Dunnett's post. C Examples of hematoxylin and eosin staining of paraffin embedded formalin fixed hearts demonstrates differences in wall morphology between Control animals and HF-saline, PNA5, and Ang-(1-7) treatment mice. Left ventricle wall is indicated with black arrow. Images were taken at 5× magnification.

FIG. 8A-B. Ang-(1-7)/MasR agonists rescue cognitive impairment in a VCID mice. A Discrimination ratio=(Time spent at new object−Time spent at familiar object)/total exploration time. Heart failure (HF) mice were treated with saline PNA5 or Ang-(1-7) at two different concentrations of either 50 ug/kg, (P50, A50) or 500 ug/kg (P500, A500). ****p<0.001 Control-Saline vs HF-Saline. *p<0.05 HF-Saline vs HF-P50, HF-P500, HF-A500. Data are represented with mean and ±SE. Difference in groups were tested by ANOVA, Dunnett's post, significance p<0.05. B No significant differences are noted in total exploration time in the familiarization test for all treatment groups. Data are represented with mean and ±SE. Significancy were tested by ANOVA.

FIG. 9A-C. Treatment with Ang-(1-7)/Mas R agonists decreases plasma NfL levels. A Data are represented with mean and ±SE. Compares NfL levels between control saline and HF-saline mice, p=0.08, Welch's t-test. This difference did not reach significance. B PNA5 at 50 ug/kg and Ang-(1-7) at 50 ug/kg, and 500 ug/kg significantly reduced NfL plasma concentration in VCID mice compared to HF-saline treated mice, Kruskal-Wallis ANOVA, Dunn's post, p<0.05. C NfL plasma levels significantly negatively correlated with Discrimination ratios. Correlation R values were produced using Pearson's correlation analysis and the fit line was determined using simple linear regression.

FIG. 10. Increased TNFα in VCID models are inhibited by PNA5 and Ang-(1-7). Plasma TNFα of Control-Saline, HF-Saline, HF-A50 (Ang-(1-7)₅₀ ug/kg), HF-P50 (PNA5 50 ug/kg), HF-A500 (Ang-(1-7)₅₀₀ ug/kg), HF-P500 (PNA5 500 ug/kg), were measured via MILLIPLEX MAP Mouse High Sensitivity Multiplex Immunoassay. Significance were observed between HF-Saline vs Control-Saline treated mice (3.50±SE 0.15, n=6 vs mean 3.12, ±SE 0.01, n=6, respectively, p=0.003), PNA5, and Ang-(1-7) (HF-P50 mean 3.12 pg/mL±SE 0.00, n=5 p=0.004; HF-A50 mean 3.12 pg/mL±SE 0.00, n=6, p=0.003; HF-P500 mean 3.12 pg/mL±SE 0.00, n=6, p=0.003; ANOVA, Dunnett's post; values that were at or below assay threshold of 3.12 pg/mL were represented as 3.12 pg/mL).

FIG. 11A-F. Effect of treatment with Ang-(1-7)/MasR agonists on brain cytokine levels. Whole brain lysate cytokine levels were measured using MILLIPLEX MAP Mouse High Sensitivity Multiplex Immunoassay. A-F HF-Saline treated VCID mice had significantly lower IL-1α, IL-2, IL-17, and IP-10 levels in comparison to Control-Saline treated mice. PNA5 and Ang-(1-7) significantly increased cytokines levels as compared to HF-saline treated animals. Data are represented with mean and ±SE. Differences in groups were tested by ANOVA, Dunnett's post hoc, significance set a p<0.05.

FIG. 12A-F. Relationship between brain cytokine levels and NfL levels. A-E The relationship between brain cytokines IL-2, IL-5, IL-13, IL-17, IP-10, and plasma NfL levels were analyzed using Pearson correlation. F Brain cytokine composite score of IL-2, IL-13, IL-17 were significantly negatively correlated with NfL plasma concentrations.

FIG. 13. Pearson's correlation matrix of the relationship between plasma NfL and brain cytokine levels. The relationship between the brain cytokines IL-2, IL-5, IL-13, IL-17, IP-10, the composite cytokine z-score and plasma NfL levels were analyzed using Pearson correlation. Blue demonstrates positive correlation and red represents negative correlation. Values closer to 1 or −1 indicate more closely correlated variables.

FIG. 14A-C. Longitudinal comparison of plasma cytokines in VCID mouse model. A-C Plasma inflammatory cytokine levels from mice with 3 weeks of HF, and 8 weeks of HF, in both Control-Saline, and HF-Saline treated mice were measured via MILLIPLEX MAP Mouse High Sensitivity Multiplex Immunoassay. At 3 weeks of HF, HF-Saline mice had significantly higher IL-1α, MIP-1α, and MIP-2 concentrations than age-matched Control-Saline treated mice. A significant decrease in cytokine concentrations was observed in HF-Saline-treated mice from 3 to 8 weeks of HF. Data are represented with mean and ±SE. Difference in groups were tested by ANOVA.

FIG. 15. Serum NfL and pTau increased with severity of heart failure.

### DEFINITIONS

The term “native Ang-(1-7)” refers to the naturally-occurring Ang(1-7) polypeptide having the amino acid sequence Asp-Arg-Val-Tyr-Ile-His-Pro (SEQ ID NO: 2).

The term “Ang-(1-7) derivative” refers to oligopeptide in which one or more amino acid residue is either modified or different than the amino acid residue of the corresponding native Ang-(1-7). The term “Ang-(1-7) derivative” also includes oligopeptide of eight amino acid residues as discussed in more detail below.

By “PN-A2” is meant the Ang(1-7) derivative of SEQ ID NO: 3, which is has the amino acid sequence of native Ang(1-7) except that Pro′ comprises a C-terminal amidation (NH₂).

By “PN-A3” is meant the Ang(1-7) derivative of SEQ ID NO: 9, which is has the amino acid sequence of native Ang(1-7) with the addition of a serine at the C-terminus (i.e., Ser⁸) and wherein Ser⁸ is glycosylated and comprises a C-terminal amidation (NH₂).

By “PN-A4” is meant the Ang(1-7) derivative of SEQ ID NO: 9, which is has the amino acid sequence of native Ang(1-7) with the addition of a serine at the C-terminus (i.e., Ser⁸) and wherein Ser⁸ is lactosylated and comprises a C-terminal amidation (NH₂).

By “PN-A5” is meant the Ang(1-7) derivative of SEQ ID NO: 13, which is has the amino acid sequence of native Ang(1-7) except that Pro′ is substituted by See and wherein See is glycosylated and comprises a C-terminal amidation (NH₂).

By “PN-A6” is meant the Ang(1-7) derivative of SEQ ID NO: 13, which is has the amino acid sequence of native Ang(1-7) except that Pro′ is substituted by See and wherein See is lactosylated and comprises a C-terminal amidation (NH₂).

The term “carbohydrate” refers to pentose and hexose of empirical formula (CH₂O)ₙ, where n is 5 for pentose and 6 for hexose. A carbohydrate can be monosaccharide, disaccharide, oligosaccharide (e.g., 3-20, typically 3-10, and often 3-5 monomeric saccharides are linked together), or polysaccharide (e.g., greater than 20 monomeric saccharide units). More often, the term carbohydrate refers to monosaccharide and/or disaccharide. However, it should be appreciated that the scope of the invention is not limited to mono- or di-saccharides. Often the terms “carbohydrate” and “saccharide” are used interchangeably herein.

The term “oligopeptide” as used throughout the specification and claims is to be understood to include amino acid chain of any length, but typically amino acid chain of about fifteen or less, often ten or less, still more often eight or less, and most often seven or eight.

It should be appreciated that one or more of the amino acids of Ang-(1-7) can be replaced with an “equivalent amino acid”, for example, L (leucine) can be replaced with isoleucine or other hydrophobic side-chain amino acid such as alanine, valine, methionine, etc., and amino acids with polar uncharged side chain can be replaced with other polar uncharged side chain amino acids. While Ang-(1-7) comprises 7 amino acids, in some embodiments the oligopeptide of the invention has eight or less amino acids.

By “glycosylated,” is meant the covalent attachment to that amino acid of a mono-, di-, or polysaccharide. The glycosylation may be N-linked or O-linked, as appropriate. For example, N-linked glycosylation may occur at the R-group nitrogen in asparagine or arginine, and β-linked glycosylation may occur through the R-group hydroxyl of serine, threonine, and tyrosine. Suitable carbohydrates include, for example, monosaccharides such as glucose, galactose, fructose, xylose, ribose, arabinose, lyxose, allose, altrose, mannose, fucose, and rhamnose, disaccharides such as sucrose, lactose, maltose, trehalose, melibiose, cellobiose, higher-order structures such as sorbitol, mannitol, maltodextrins, and farinose, and amino sugars such as galactosamine and glucosamine. In some particular embodiments, the polypeptide is glycosylated with glucose, lactose, cellobiose, melibiose, β-D-glucose, β-D-lactose, β-D-cellobiose, or β-D-melibiose.

The term “combinations thereof,” which reference to any modifications (e.g, carbohydrate modifications) of Ang-(1-7) derivatives refers to oligopeptides in which two, three, four, five, six, seven, or eight of the individual amino acids are modified by the attachment of a carbohydrate. For Ang-(1-7) derivatives having a plurality of carbohydrate modifications, the modifying carbohydrates may be the same on every modified amino acid, or the several modified amino acids may comprise a mixture of different carbohydrates.

“A therapeutically effective amount” means the amount of a compound that, when administered to a mammal, at an appropriate interval and for a sufficient duration for treating a disease, is sufficient to effect such treatment for the disease. The “therapeutically effective amount” will vary depending on the compound, the disease and its severity, physiological factors unique to the individual including, but not limited to the age, weight, and body mass index, the unitary dosage, cumulative dosage, frequency, duration, and route of administration selected.

“Prevent,” when used in connection with the occurrence of a disease, disorder, and/or condition, refers to reducing the risk of developing the disease, disorder and/or condition for which the subject is at risk of developing.

The terms “treat” or “treating” refer to any method used to partially or completely alleviate, ameliorate, relieve, inhibit, prevent, reduce severity of one or more symptoms or features of a particular disease, disorder, and/or condition in a subject diagnosed as having that disease or disorder. For example, treatment of a cognitive dysfunction and/or impairment may refer to: (1) preventing cognitive dysfunction and/or impairment from occurring, i.e., causing the clinical symptoms of cognitive dysfunction and/or impairment not to develop in a subject that may be or predisposed to developing cognitive dysfunction and/or impairment but does not yet experience or display symptoms of cognitive dysfunction and/or impairment; (2) inhibiting cognitive dysfunction and/or impairment, i.e., arresting or reducing the development of cognitive dysfunction and/or impairment or its clinical symptoms; or (3) relieving cognitive dysfunction and/or impairment, i.e., causing regression of cognitive dysfunction and/or impairment or its clinical symptoms.

Symptoms of cognitive dysfunction or cognitive impairment may include, but are not limited to progressive loss of memory, cognition, reasoning, judgment, aspects of higher cortical function, diminished initiative, excessive distraction, speech, motor activity, recognition of perceptions, exaggerated or caricatured personality traits, irritability, excessive anger, violence, uncontrollable agitation, and delusions.

The terms “approximately” or “about” in reference to a number are generally taken to include numbers that fall within a range of 5%, 10%, 15%, or 20% in either direction (greater than or less than) of the number unless otherwise stated or otherwise evident from the context (except where such number would be less than 0% or exceed 100% of a possible value).

The term “subject” or “patient” refers to any organism to which a composition of this invention may be administered, e.g., for experimental, diagnostic, and/or therapeutic purposes. Typical subjects include animals (e.g., mammals such as mice, rats, rabbits, dogs, cats, non-human primates, and humans).

By “dosing regimen” is meant a set of unit doses (e.g., one, two, three, four, or more) that is/are administered individually to a subject, typically separated by periods of time. In some embodiments, a dosing regimen comprises one or a plurality of doses each of which are separated from one another by a time period. The time period separating individual doses may have a fixed or variable duration, or the therapeutic agent may be administered on an as-need basis. A dosing regimen may span one day, multiple days, multiple weeks, multiple months, or be administered for the lifetime of the subject (e.g., 1, 2, 3, 4, 5, 6, 7, 10, 14, 21, or 28 days, or 1, 2, 3, 4, 5, 6, 9, or 12 months or more). In some embodiments, the therapeutic agent is administered once a day (QD), twice a day (BID), three times a day (TID), four times a day (QID), or less frequently (i.e., every second or third day, one each week, or once each month).

By “reference level,” when referring to a concentration of NFL and/or p-tau (e.g., p-tau181) in a biological sample obtained from a subject is meant a concentration above which is indicative of a disease or condition of interest and/or below which is indicative of the effectiveness of a therapy. The selected reference level may be (i) an absolute amount, (ii) an amount based on the NFL and/or p-tau (e.g., p-tau181) level measured in a reference population including, for example, subjects identified as not having the disease or condition of interest or subjects that have not received the treatment of interest, or (iii) an amount from the same individual measured prior to an intervention or event including, for example, a pre-treatment or pre-surgical level. For example, a serum NFL level greater than about 25, 30, 35, 40, 45, 50, 60, 65, 70, or 75 pg/ml is indicative of active nervous system injury including, for example, Alzheimer's disease and cognitive dysfunction such as VCI and post-surgical cognitive impairment. Alternatively, when used to monitor therapeutic efficacy, a subject may serve as his/her own control wherein a pre-therapy NFL level is measured and a significant reduction in the NFL and/or p-tau (e.g., p-tau181) level following therapy indicates therapeutic efficacy.

Unless defined otherwise, all technical and scientific terms used herein have the meaning commonly understood by a person skilled in the art to which this invention belongs.

### DETAILED DESCRIPTION OF THE INVENTION

Experiments conducted during the course of developing embodiments for the present invention demonstrated that 1) blood-based measurements of serum neurofilament light protein (NFL) is increased in a murine model of VCID as well as in patients with diagnosed VCID, and 2) treatment with Ang-(1-7) derivative oligopeptides (e.g., PN-A5) modifies NFL and is correlated with levels of brain inflammatory cytokines and cognitive dysfunction.

Indeed, additional experiments demonstrated that treatment with Ang-(1-7)/MasR agonists reversed VCID-induced cognitive impairment and significantly decreased NfL levels in a mouse model of VCID as compared to HF-saline treated mice. Further, NfL levels were shown to be significantly negatively correlated with cognitive scores and the concentrations of multiple pleiotropic cytokines in the brain. Such results indicate that treatment with Ang-(1-7)/MasR agonists rescues cognitive impairment and decreases plasma NfL relative to HF-saline-treated animals in a VCID mouse model.

Additional experiments demonstrated increases in circulating neurodegeneration biomarkers neurofilament light protein and phosphorylated tau181 (e.g., p-tau181) in individuals with preserved cardiac function heart failure. Moreover, such experiments further demonstrated that serum NfL and pTau increased with severity of heart failure.

Accordingly, the present invention provides compositions and methods useful in the diagnosis and treatment of a variety of cognitive impairments including vascular cognitive impairment and dementia, and Alzheimer's Disease. In one particular embodiment, the level of the neurofilament light protein and/or phosphorylated tau (e.g., p-tau181) is used to diagnose a cognitive impairment or monitor the efficacy of a therapy.

Neurofilament light protein (NFL) is a neuronal cytoplasmic protein highly expressed in large myelinated axons. NFL is one of three subunits that make up neurofilaments which are major components of the neuronal cytoskeleton. It has been observed that the NFL level is increased in cerebrospinal fluid (CSF) and blood proportionally to the degree of axonal damage in several neurological disorders. The present inventions are based, in part, on the discovery that NFL is a sensitive biomarker for VCI and AD, as well as the progression and severity of those diseases. Furthermore, it was discovered NFL levels are reduced in response to effective therapies.

Tau protein has a causative role in Alzheimer's disease and multiple other neurodegenerative disorders exhibiting tau histopathology collectively termed tauopathies. The primary function of tau protein is to facilitate assembly and maintenance of microtubules in neuronal axons. In the disease process tau protein becomes modified, loses its affinity to microtubules and accumulates in the cell body where it forms aggregates. The large neurofibrillary tangles formed from tau protein assembled into filaments were thought to be the pathological structure of tau.

Cognitive dysfunction or impairment is a common neurological complication of impaired blood flow to the brain which may be caused by a variety of conditions including stroke, atherosclerosis, congestive heart failure (“CHF”), and cardiac surgery. Without being bound by any theory, it is believed that in general any clinical condition associated with an increase in inflammatory cytokines and/or increase in reactive oxygen species (ROS) in central nervous system, in particular in the brain, can lead to cognitive dysfunction. Many of these biochemical features also are observed during the progression of Alzheimer's disease.

The role of ROS in learning and memory has been extensively studied. All of the NAD(P)H oxidase subunits, including NOX2 and NOX4, have been localized within the cell bodies and dendrites of neurons of the mouse hippocampus and perirhinal cortex and are co-localized at synaptic sites. These are key regions of the brain in learning and memory. In the brain, superoxide production via actions of NAD(P)H oxidase are known to be involved in neurotoxicity, age related dementia, stroke and neurodegenerative diseases and have been identified throughout the brain including the hippocampus, thalamus, cerebellum and amygdala. In younger, healthy animals ROS and NAD(P)H oxidase is shown to be required for normal learning and hippocampal long-term potentiation (LTP). Recent studies in mice lacking Mas have shown that Ang-(1-7) and Mas are essential for normal object recognition processing and blockade of Mas in the hippocampus impairs object recognition. In addition, Ang-(1-7) facilitates LTP in CA1 cells and this effect is blocked by antagonism of Mas.

Over the last decade, it has become recognized that renin angiotensin system (RAS) involves two separate enzymatic pathways providing a physiological counterbalance of two related peptides acting at distinct receptors. The well described ACE-AngII-AT1 receptor system is thought to be physiologically opposed and balanced by the ACE2-Ang-(1-7)-Mas system. Functionally, these two separate enzymatic pathways of RAS are thought to be involved in balancing ROS production and nitric oxide (NO) in the brain, microvasculature and peripheral tissues. Increases in AT1 receptor activation are known to increase NAD(P)H oxidase and ROS generation which are both known to contribute to abnormal increases of sympathetic nerve activity observed in CHF and hypertension. This increase in AT1 receptor-induced ROS formation is thought to be opposed by ACE2-Ang-(1-7)-Mas inhibition of ROS formation. Ang-(1-7), the majority of which is produced from ACE2 cleavage of Ang II, decreases ROS production and increases NOS in the brain via activation Mas and, possibly through AT2 receptor.

Within the brain, the Mas receptor is known to be expressed on neurons, microglia and vascular endothelial cells. Further, all three of these key components that make up the “neurovascular unit” (neurons, microglia and endothelial cells) are central players in neurogenic hypertension and CHF-induced increases in brain inflammation and ROS production. Both CHF and hypertension increase circulating cytokines promoting ROS production within the “neurovascular unit”. The end-result of this feed-forward cascade is neuronal dysfunction and cognitive impairment. The ideal therapeutic candidate to treat cognitive impairment would be designed to interrupt this cascade by working at both sides of the blood-brain barrier, the brain vascular endothelium and neuronal cells. Ang-(1-7), acting at the Mas receptor, is known to have effects at both endothelial cells and neurons. However, using a native Ang-(1-7) for treating cognitive dysfunction and/or impairment is not suitable because native Ang-(1-7) is susceptible to enzymatic degradation. Moreover, native Ang-(1-7) does not readily cross the blood-brain barrier to be suitable as a therapeutic agent.

Without being bound by any theory, it is believed that one of the advantages of using Ang-(1-7) derivatives for treating cognitive dysfunction and/or impairment is that the Ang-(1-7) derivatives have enhanced endothelial “interaction” and brain penetration. It is believed that the Ang-(1-7) derivatives act at both endothelial cells and neurons thus inhibiting inter alia neurovascular ROS production and mitigating the brain inflammatory cascade.

The renin-angiotensin system (RAS), well known for roles in blood pressure regulation and fluid homeostasis, was recently implicated in metastatic bone disease including inflammation, angiogenesis, tumor cell proliferation, and migration. Angiotensin II (Ang II) is the major end product of the RAS through cleavage by Angiotensin Converting Enzyme (ACE). This nonapeptide binds to and activates two G-protein coupled receptors (GPCRs): angiotensin II receptor type 1 (AT1) and type 2 (AT2). Physiological effects such as vasoconstriction, inflammation, fibrosis, cellular growth/migration, and fluid retention are reported for AT1 and AT2. Ang II is cleaved by ACE2 to yield Angiotensin-(1-7) (Ang-(1-7)), a biologically active heptapeptide. In contrast to Ang II, Ang-(1-7) binds to the GPCR, Mas receptor (MasR; Kd=0.83 nM) with 60-100 fold greater selectivity over the AT1 and AT2 receptors. Activation of the MasR elicits effects opposite to those of the Ang II/AT1/AT2 axis including having anti-inflammatory and antidepressant activities.

Some aspects of the invention includes the use of oligopeptides that are derivatives of Ang-(1-7). As discussed above, the term “derivative” of Ang-(1-7) refers to an oligopeptide whose amino acid sequence of any one or more of Ang-(1-7) is modified (e.g., via methylation, presence of a functional group, such as hydroxy group on proline), attached to a carbohydrate, is replaced with corresponding D-amino acid or an “equivalent amino acid” as defined above, and/or the terminal amino group end or the carboxyl end of Ang-(1-7) is modified, for example, the carboxylic acid end can be modified to be an amide, an amine, a thiol, or an alcohol functional group, or one in which an additional amino acid residue is present compared to native Ang-(1-7). It should be appreciated that the term “Ang-(1-7) derivative” excludes the native Ang-(1-7), i.e., amino acid sequences of endogenous Ang-(1-7) without any modification.

In some embodiments, oligopeptides of the invention have the amino group on the carboxylic acid terminal end (i.e., the —OH group of the carboxylic acid is replaced with —NR^{a}R^{b}, where each of Ra and R b is independently hydrogen or C₁-C₆ alkyl) and/or have one or more amino acid residues that are (i) replaced with a corresponding D-amino acid, (ii) glycosylated, (iii) replaced with another amino acid, (iv) or a combination thereof.

In one particular embodiment, the oligopeptide of the invention is Ang-(1-7) derivative of the formula: A¹-A²-A³-A⁴-A⁵-A⁶-A⁷-A⁸ (SEQ ID NO:1), where A¹ is selected from the group consisting of aspartic acid, glutamic acid, alanine, and a derivative thereof; A² is selected from the group consisting of arginine, histidine, lysine, and a derivative thereof; A³ is selected from the group consisting of valine, alanine, isoleucine, leucine, and a derivative thereof; A⁴ is selected from the group consisting of tyrosine, phenylalanine, tryptophan, and a derivative thereof; A⁵ is selected from the group consisting of isoleucine, valine, alanine, leucine, and a derivative thereof; A⁶ is selected from the group consisting of histidine, arginine, lysine, and a derivative thereof; A⁷ is selected from the group consisting of proline, glycine, serine, and a derivative thereof; and A⁸ can be present or absent, wherein when A⁸ is present, A⁸ is selected from the group consisting of serine, threonine, hydroxyproline, and a derivative thereof, provided (i) at least one of A¹-A⁸ is optionally substituted with a mono- or di-carbohydrate; or (ii) when A⁸ is absent: (a) at least one of A¹-A⁷ is substituted with a mono- or di-carbohydrate, (b) A⁷ is terminated with an amino group, or (c) a combination thereof.

In some embodiments, A¹ is the amino terminal end of the oligopeptide and A⁸ (or A⁷ when A⁸ is absent) is the carboxyl terminal end. Still in other embodiments, A¹ is the carboxyl terminal end and A⁸ (or A⁷ when A⁸ is absent) is the amino terminal end. Yet in other embodiments, the carboxylic acid functional group of the carboxyl terminal end is modified as an amide functional group, an amine functional group, a hydroxyl functional group, or a thiol functional group. The amide and the amine functional groups can be non-alkylate, mono-alkylated or di-alkylated.

Yet in other embodiments, the carbohydrate comprises glucose, galactose, xylose, fucose, rhamnose, or a combination thereof. In some instances, the carbohydrate is a mono-carbohydrate, whereas in other instances, the carbohydrate is a di-carbohydrate.

In other embodiments, at least one of A¹-A⁸ is substituted with a mono-carbohydrate. Still in other embodiments, at least one of A¹-A⁸ is substituted with a di-carbohydrate. It should be appreciated that the scope of the invention also includes those oligopeptides having both mono- and di-carbohydrates.

Exemplary di-carbohydrates that can be used in oligopeptides of the invention include, but are not limited to, lactose, cellobiose, melibiose, and a combination thereof. However, it should be appreciated that the scope of the invention includes oligopeptides that are substituted with any dicarbohydrates known to one skilled in the art.

In one particular embodiment, A⁸ is serine or a derivative thereof. In some instances, the carboxylic acid moiety of the serine is modified as an amide or an amine. In one case, serine is terminated as an amino group. Still in other embodiments, the serine residue of A⁸ is glycosylated with glucose or lactose.

Yet in other embodiments, at least one, typically at least two, generally at least three, often at least four, still more often at least five, yet still more often at least six, and most often all of A¹-A⁸ is D-amino acid.

Another aspect of the invention includes the use of oligopeptides, such as Ang-(1-7) derivatives, having eight amino acids or less, typically seven or eight amino acid residues. In some embodiments, one or more amino acids have attached thereto a carbohydrate group. Often the carbohydrate group is attached to the oligopeptide via glycosylation. The carbohydrate can be attached to the oligopeptide via any of the side chain functional group of the amino acid or the amide group. Accordingly, the scope of the invention includes, but is not limited to, O-glycosylate, N-glycosylate, S-glycosylated oligopeptides. The term “X-glycosylated” refers to having a carbohydrate attached to the oligopeptide via the heteroatom “X” of the amino acid. For example, for serine whose side-chain functional group is hydroxyl, “O-glycosylated” means the carbohydrate is attached to the serine's side-chain functional group, i.e., the hydroxyl group. Similarly, “N-glycosylation” of leucine refers to having the carbohydrate attached to the amino side-chain functional group of leucine. Typically, the glycosylation is on the side-chain functional group of the amino acid.

In some embodiments, the Ang-(1-7) derivative is glycosylated with xylose, fucose, rhamnose, glucose, lactose, cellobiose, melibiose, or a combination thereof.

Yet in other embodiments, the carboxylic acid terminal end of said glycosylated Ang-(1-7) derivative is substituted with an amino group. When referring to the carboxyl acid terminal end being substituted with an amino group, it means —OH group of the carboxylic acid is replaced with —NH₂ group. Thus, the actual terminal end functional group is an amide, i.e., rather than having the oligopeptide being terminated at the carboxylic acid terminal end with a functional group —CO₂H, the carboxylic acid terminal end is terminated with an amide group (i.e., —CO₂NR′₂, where each R′ is independently hydrogen or C₁-C₁₂ alkyl). Still in other embodiments, the carboxylic acid terminal group is terminated with a hydroxyl or a thiol group. In some embodiments, the modified carboxylic acid terminal group is used to attach the carbohydrate, e.g., via glycosylation.

In some embodiments, the Ang-(1-7) derivatives have enhanced efficacy of action, in vivo stabilization, and/or penetration of the blood-brain barrier relative to native Ang-(1-7). Improved penetration of the blood-brain barrier facilitates cerebral entry of the Ang-(1-7) derivative and, consequently, Mas activation, or intrinsic-efficacy. To improve (i.e., increase) penetration of the blood-brain barrier, in some embodiments the Ang-(1-7) derivative is attached to at least one mono- or di-carbohydrates.

Without being bound by any theory, it is believed that glycosylated Ang-(1-7) derivatives exploit the inherent amphipathicity of the folded Ang-(1-7) glycopeptides (i.e., glycosylated oligopeptides of the invention) and the “biousian approach” to deliver the glycosylated oligopeptides of the invention across the blood-brain barrier. In some instances, the amount of increase in crossing the blood-brain barrier by oligopeptides of the invention is at least 6%, typically at least 10%, and often at least 15% compared to native Ang-(1-7). In some instances, the amount of increase in the Cmax for oligopeptides of the invention in cerebral-spinal fluid is 2-10 fold, 3-8 fold, or 5-8 fold compared to native Ang-(1-7). In some instances, the amount of increase in the Cmax for oligopeptides of the invention in cerebral-spinal fluid is 2, 3, 4, 5, 6, 7, 8, 9 or 10 fold compared to native Ang-(1-7). In other instances, the Ang-(1-7) derivatives have in vivo half-life of at least 20 min, at least 30 min, at least 40 min, at least 50 min, at least 60 min, or at least 2, hours, at least 3 hours, at least 4 hours, at least 5 hours or at least 6 hours. In some instances, the amount of increase in the in vivo half-life for the Ang-(1-7) derivatives is 2-30 fold, 3-25 fold, 4-20 fold, 4-10 fold, 10-25 fold, 15-25 fold, or 20-25 fold compared to native Ang-(1-7). Alternatively, compared to native Ang-(1-7), the Ang-(1-7) derivatives exhibit at least 50 fold, typically at least 75 fold, and often at least 100 fold increase in in vivo half-life.

In other embodiments, the Ang-(1-7) derivatives exhibit enhanced vascular efficacy. Without being bound by any theory, it is generally recognized that blood-brain barrier transport occurs via an absorptive endocytosis process on the blood side of the endothelium of the brain capillaries followed by exocytosis on the brain side, leading to overall transcytosis. It is also believed that for this process to be efficient, the oligopeptide must bind to the membrane for some period of time, and must also be able to exist in the aqueous state for some period of time (biousian nature). Based on previous work from one of the present inventors, it is believed that effective drug delivery and blood-brain barrier transport requires a biousian glycopeptide that has at least two states: (1) a state defined by one or more membrane-bound conformations that permit or promote endocytosis; and (2) a state defined by a water-soluble, or random coil state that permits “membrane hopping” and, presumably, vascular efficacy.

In general, the degree of glycosylation does not have a large effect on the structure of the individual microstates. Thus, altering the degree of glycosylation allows for the modulation of aqueous vs. membrane-bound state population densities without significantly affecting the overall structure of the oligopeptide. Moreover, it is believed that glycosylation also promotes stability to peptidases, thereby increasing the half-life of the Ang-(1-7) derivatives in vivo. TABLE 1 sets forth some particularly useful Ang(1-7) derivative polypeptides but is not intended to be limiting on the scope of the invention.

TABLE 1

| Amino Acid Position | | | | | | | | SEQ |
|---|---|---|---|---|---|---|---|---|
| 1 | 2 | 3 | 4 | 5 | 6 | 7 | 8 | ID NO: |
| Asp | Arg | Val | Tyr | Ile | His | Pro | — | 2 |
| Asp | Arg | Val | Tyr | Ile | His | Pro° | — | 3 |
| Asp | Arg | Val | Tyr | Ile | His | Pro* | — | 4 |
| Asp | Arg | Val | Tyr | Ile | His | Pro°* | — | 5 |
| Asp | Arg | Val | Tyr | Ile | His | Pro | Ser | 6 |
| Asp | Arg | Val | Tyr | Ile | His | Pro | Ser° | 7 |
| Asp | Arg | Val | Tyr | Ile | His | Pro | Ser* | 8 |
| Asp | Arg | Val | Tyr | Ile | His | Pro | Ser°* | 9 |
| Asp | Arg | Val | Tyr | Ile | His | Ser | — | 10 |
| Asp | Arg | Val | Tyr | Ile | His | Ser° | — | 11 |
| Asp | Arg | Val | Tyr | Ile | His | Ser* | — | 12 |
| Asp | Arg | Val | Tyr | Ile | His | Ser°* | — | 13 |
| Ala | Arg | Val | Tyr | Ile | His | Pro | — | 14 |
| Ala | Arg | Val | Tyr | Ile | His | Pro° | — | 15 |
| Ala | Arg | Val | Tyr | Ile | His | Pro* | — | 16 |
| Ala | Arg | Val | Tyr | Ile | His | Pro°* | — | 17 |
| Ala | Arg | Val | Tyr | Ile | His | Pro | Ser | 18 |
| Ala | Arg | Val | Tyr | Ile | His | Pro | Ser° | 19 |
| Ala | Arg | Val | Tyr | Ile | His | Pro | Ser* | 20 |
| Ala | Arg | Val | Tyr | Ile | His | Pro | Ser°* | 21 |
| Ala | Arg | Va | Tyr | Ile | His | Ser | — | 22 |
| Ala | Arg | Val | Tyr | Ile | His | Ser° | — | 23 |
| Ala | Arg | Va | Tyr | Ile | His | Ser* | — | 24 |
| Ala | Arg | Val | Tyr | Ile | His | Ser°* | — | 25 |
| Asp | Arg | Nle | Tyr | Ile | His | Pro | — | 26 |
| Glu | Lys | Val | Ser | Va | Arg | Ser | | |
| Ala | Ala | Leu | Thr | Leu | — or ° | Cys | | |
| Asn | — or ° | Ile | Ala | Nle | | —, °, * | | |
| Pro | | Ala | — or ° | Ala | | or °* | | |
| Gly | | Gly | | Gly | | | | |
| — or ° | | Lys | | | | | | |
| | | Pro | | | | | | |
| | | Tyr | | | | | | |
| | | — or ° | | | | | | |
| Asp | Arg | Nle | Tyr | Ile | His | Pro | Phe | 27 |
| Glu | Lys | Val | Ser | Val | Arg | Ala | Ser | |
| Ala | Ala | Leu | Thr | Leu | — or ° | | Cys | |
| Asn | — or ° | Ile | Ala | Nle | | | Ile | |
| Pro | | Ala | — or ° | Ala | | | Tyr | |
| Gly | | Gly | | Gly | | | —, °, *, | |
| — or ° | | Lys | | | | | or °* | |
| | | Pro | | | | | | |
| | | Tyr | | | | | | |
| | | — or ° | | | | | | |

¹ - Where more than one amino acid is indicated, the amino acids are presented in the alternative.
— = unmodified
° = glycosylated
* = carboxy terminal NH₂

In some embodiments, only the C-terminal amino acid is glycosylated (i.e., Xaa⁸ or Xaa⁷ if Xaa⁸ is absent). In some embodiments, the Ang(1-7) derivative polypeptide is glycosylated with glucose, lactose, cellobiose, melibiose, β-D-glucose, β-D-lactose, β-D-cellobiose, or β-D-melibiose. In some embodiments, the polypeptide comprises an O-linked glycosylation (e.g., on the R-group of a serine). In some embodiments, the C-terminal serine is glycosylated.

In some embodiments, non-naturally-occurring amino acids and/or amino acid substitutes (e.g., dicarboxylic acids) may be substituted for the naturally-occurring amino acids in Ang(1-7) and any of the Ang(1-7) derivative polypeptides including, for example, in the Ang(1-7) derivative polypeptides of TABLE 1. For example, α,α-disubstituted amino acids, N-alkyl amino acids, C-α-methyl amino acids, β-amino acids, and β-methyl amino acids. Amino acids analogs useful in the present invention may include, but are not limited to, β-alanine, norvaline, norleucine, 4-aminobutyric acid, orithine, hydroxyproline, sarcosine, citrulline, cysteic acid, cyclohexylalanine, 2-aminoisobutyric acid, 6-aminohexanoic acid, t-butylglycine, phenylglycine, o-phosphoserine, N-acetyl serine, N-formylmethionine, 3-methylhistidine and other unconventional amino acids. For example,
Xaa¹ may be Acpc (1-aminocyclopentane carboxylic acid), Me2Gly (N,N-dimethylglycine), Bet (betaine, 1-carboxy-N,N,N-trimethylmethanaminium hydroxide), Sar (sarcosine) or Suc (succinic acid);
Xaa² may be Cit (citrulline), Orn (ornithine), acetylated Ser, or Sar;
Xaa³ may be Nle (norleucine), hydroxyproline, Acpc, or Aib (2-aminoisobutyric acid);
Xaa⁴ may be Tyr(P03), homoserine, azaTyr (aza-α¹-homo-L-tyrosine);
Xaa⁵ may be Nle, hydroxyproline, Acpc, or Aib;
Xaa⁶ may be 6-NH₂-Phe (6-aminophenylalaine); and
Xaa⁸ may be Phe(Br) (p-bromo-phenylalanine; may be L- or D-phenylalanine).

In some embodiments, the Ang(1-7) derivative polypeptide does not comprise the naturally-occurring amino acid sequence of native Ang(1-7) set forth in SEQ ID NO: 2.

In some embodiments, Ang(1-7) and any of the Ang(1-7) derivative polypeptides, including those specifically defined in TABLE 1, may comprise entirely L-amino acids, entirely D-amino acids, or a mixture of L- and D-amino acids (e.g., having 1, 2, 3, 4, 5, 6, 7, or 8 D-amino acids).

The Ang(1-7) and Ang(1-7) derivative polypeptides may be produced by any suitable method including, without limitation, by peptide synthesis methods such exclusive solid phase synthesis, partial solid phase synthesis, fragment condensation, classical solution synthesis, native-chemical ligation, and recombinant techniques.

Ang-(1-7) derivatives can be administered to a patient to achieve a desired physiological effect. Preferably the patient is an animal, more preferably a mammal, and most preferably a human. The oligopeptide can be administered in a variety of forms adapted to the chosen route of administration, i.e., orally or parenterally. Parenteral administration in this respect includes administration by the following routes: intravenous; intramuscular; subcutaneous; intraocular; intrasynovial; transepithelially including transdermal, ophthalmic, sublingual and buccal; topically including ophthalmic, dermal, ocular, rectal and nasal inhalation via insufflation and aerosol; intraperitoneal; and rectal systemic.

The active oligopeptide can be orally administered, for example, with an inert diluent or with an assimilable edible carrier, or it can be enclosed in hard or soft shell gelatin capsules, or it can be compressed into tablets. For oral therapeutic administration, the active oligopeptide may be incorporated with excipient and used in the form of ingestible tablets, buccal tablets, troches, capsules, elixirs, suspensions, syrups, wafers, and the like. Such compositions and preparation can contain at least 0.1% of active oligopeptide. The percentage of the compositions and preparation can, of course, be varied and can conveniently be between about 1 to about 10% of the weight of the unit. The amount of active oligopeptide in such therapeutically useful compositions is such that a suitable dosage will be obtained. Preferred compositions or preparations according to the present invention are prepared such that an oral dosage unit form contains from about 1 to about 1000 mg of active oligopeptide.

The tablets, troches, pills, capsules and the like can also contain the following: a binder such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, lactose or saccharin can be added or a flavoring agent such as peppermint, oil of wintergreen, or cherry flavoring. When the dosage unit form is a capsule, it can contain, in addition to materials of the above type, a liquid carrier. Various other materials can be present as coatings or to otherwise modify the physical form of the dosage unit. For instance, tablets, pills, or capsules can be coated with shellac, sugar or both. A syrup or elixir can contain the active oligopeptide, sucrose as a sweetening agent, methyl and propylparabens a preservatives, a dye and flavoring such as cherry or orange flavor. Of course, any material used in preparing any dosage unit form should be pharmaceutically pure and substantially non-toxic in the amounts employed. In addition, the active oligopeptide can be incorporated into sustained-release preparations and formulation.

The active oligopeptide can also be administered parenterally. Solutions of the active oligopeptide can be prepared in water suitably mixed with a surfactant such as hydroxypropylcellulose. Dispersion can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It can be stable under the conditions of manufacture and storage and must be preserved against the contaminating action of microorganisms such as bacterial and fungi. The carrier can be a solvent of dispersion medium containing, for example, water, ethanol, polyol (e.g., glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, e.g., sugars or sodium chloride. Prolonged absorption of the injectable compositions of agents delaying absorption, e.g., aluminum monostearate and gelatin.

Sterile injectable solutions are prepared by incorporating the active oligopeptide in the required amount in the appropriate solvent with various other ingredients enumerated above, as required, followed by filtered sterilization. Generally, dispersions are prepared by incorporating the various sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze-drying technique which yield a powder of the active ingredient plus any additional desired ingredient from previously sterile-filtered solution thereof.

The therapeutic Ang-(1-7) derivatives can be administered to a mammal alone or in combination with pharmaceutically acceptable carriers, as noted above, the proportion of which is determined by the solubility and chemical nature of the oligopeptide, chosen route of administration and standard pharmaceutical practice.

Additional objects, advantages, and novel features of this invention will become apparent to those skilled in the art upon examination of the following examples thereof, which are not intended to be limiting. In the Examples, procedures that are constructively reduced to practice are described in the present tense, and procedures that have been carried out in the laboratory are set forth in the past tense.

### EXPERIMENTAL

The following examples are illustrative, but not limiting, of the present invention. Other suitable modifications and adaptations of the variety of conditions and parameters normally encountered in clinical therapy and which are obvious to those skilled in the art are within the spirit and scope of the invention. As used herein, terms such as “our”, “we”, “I”, and similar terms, refers to the inventive entity for the inventions described herein.

### Example 1: Murine Model of Heart Failure

The purpose of the present study was to 1) determine whether blood-based measurements of serum neurofilament light protein (NFL) is increased in a murine model of VCID as well as in patients with diagnosed VCID and 2) determine whether treatment with Ang-(1-7) derivative oligopeptides (e.g., PN-A5) modifies NFL and is correlated with levels of brain inflammatory cytokines and cognitive dysfunction.

Heart Failure Model And Experimental Design: The sham group received treatment with saline vehicle. The HF group was divided into five (5) treatment groups as follows:

Male C57Bl/6J adult mice (Harlan, 8-10 weeks old) were randomly assigned to either the sham control (n=12) or heart failure (HF) group (n=44). All mice prior to surgery were weighed and anesthetized. For the HF mice, myocardial infarction (MI) was induced by ligation of the left coronary artery (LCA). Under anesthesia (2.5% isoflurane in a mixture of air and 02) a thoracotomy was performed at the fourth left intercostal space and the LCA permanently ligated to induce a myocardial infarction (MI). Occlusion of the LCA was confirmed by observing blanching, a slight change in color of the anterior wall of the left ventricle downstream of the ligature. Sham mice underwent the same procedure with the exception of ligating the LCA.

Following 5 weeks post MI surgery, mice were treated for 21 days with daily subcutaneous injections as follows:

| | Number of | |
|---|---|---|
| Group | Subjects (n) | Treatment |
| Control-saline | 12 | Saline (vehicle control) |
| HF-saline | 12 | Saline (vehicle control) |
| HF-A50 | 8 | Ang-(1-7), 50 μg/kg/day |
| HF-P50 | 6 | PN-A5, 50 μg/kg/day |
| HF-A500 | 6 | Ang-(1-7), 500 μg/kg/day |
| HF-P500 | 12 | PN-A5, 500 μg/kg/day |

Following treatment, animals were tested for object recognition using a standard NOR test as described below. Animals then were sacrificed, and serum and blood samples were obtained for further analysis.

Novel Object Recognition (NOR): The novel object recognition (NOR) task, as it pertains to the study of working memory and attention, is predicated on rodent preference of novel stimuli, whether spatial or otherwise (Ennaceur, Cavoy, Costa, & Delacour, 1989; Ennaceur & Delacour, 1988; Goulart et al., 2010; Silvers, Harrod, Mactutus, & Booze, 2007). When novel objects are paired simultaneously with familiar ones in an environment to which the animal has been habituated, it is possible to use the difference in exploration times of each object to make determinations of the degree of cognitive impairment relative to a measured baseline (Aggleton, Albasser, Aggleton, Poirier, & Pearce, 2010; Antunes & Biala, 2012; Olarte-Sanchez, Amin, Warburton, & Aggleton, 2015). The primary metric used to compare mice of different groups is the discrimination ratio (DR)—a value calculated as the ratio of time spent exploring the novel object (NO) to the total time spent exploring the familiar objects (FO) in addition the NO, i.e. DR=Time at NO/(Time at NO+Time at FO).

The apparatus consisted of an evenly illuminated Plexiglas box (12 cm×12 cm×12 cm) placed on a table inside an isolated observation room. All walls of the apparatus were covered in black plastic, and the floor was grey with a grid that was used to ensure that the location of objects did not change between object familiarization and test phases. The mouse behavior and exploration of objects was recorded with a digital camera. The digital image from the camera was fed into a computer in the adjacent room. Two digital stopwatches were used to track the time the mouse spent interacting with the objects of the test. All data was downloaded to Excel files for analysis. Triplicate sets of distinctly different objects were used for the test.

The novel object recognition task included 3 phases: habituation phase, familiarization phase, and test phase. For the habituation phase, on the first and second day, mice were brought to the observation room habituated to the empty box for 10 min per day. On the third day, each mouse had a “familiarization” trial with two identical objects followed by a predetermined delay period and then a “test” trial in which one object was identical to the one in the familiarization phase, and the other was novel. All stimuli were available in triplicate copies of each other so that no object needed to be presented twice. Objects were made of glass, plastic or wood that varied in shape, color, and size. Therefore, different sets of objects were texturally and visually unique. Each mouse was placed into the box the same way for each phase, facing the center of the wall opposite to the objects. To preclude the existence of olfactory cues, the entire box and objects were always thoroughly cleaned with 70% ethanol after each trial and between mice. During the familiarization phase, mice were allowed to explore the two identical objects for 4 min and then returned to their home cages. After a 2 hour delay, the “test phase” commenced. The mice were placed back to the same box, where one of the two identical objects presented in the familiarization phase was switched to a novel one and the mouse was allowed to explore these objects for another 4 min. Mouse “exploratory behavior” was defined as the animal directing its nose toward the object at a distance of ˜2 cm or less. Any other behavior, such as resting against the object, or rearing on the object was not considered to be exploration. Exploration was scored by an observer blind to the mouse's surgical and treatment group. Finally, the positions of the objects in the test phases, and the objects used as novel or familiar, were counterbalanced among the groups of mice.

Discrimination ratios were calculated from the time spent exploring the novel object minus time spent exploring the familiar object during the test phase divided by the total exploration time. DRatio=(t novel−t familiar)/(t novel+t familiar). Data were analyzed from first 2 minutes of ‘test phase’. A positive score indicates more time spent with the novel object, a negative score indicates more time spent with the familiar object, and a zero score indicates a null preference. All NOR data was examined using one-way analysis of variance, between subjects (ANOVA). Individual group differences were tested using the post hoc Tukey HSD test. In comparisons between groups of different sample sizes, equal variance was tested using a modified Levene's test. All statistical tests and p-values were calculated using MS Excel with Daniel's XLtoolbox and alpha was set at the 0.05 level. Error bars represent SEM.

NFL Quantification: Immediately following the final NOR test, mice were sacrificed and serum samples were obtained for NFL quantification using the Simoa™ NF-Light® kit (Quanterix Cat #103186, Lot #501953), which quantifies neurofilament light (NFL). Assay samples were diluted 1:4 and calibrators were undiluted. As samples and calibrators were run in duplicate and analyzed using the HD-X Analyzer software package.

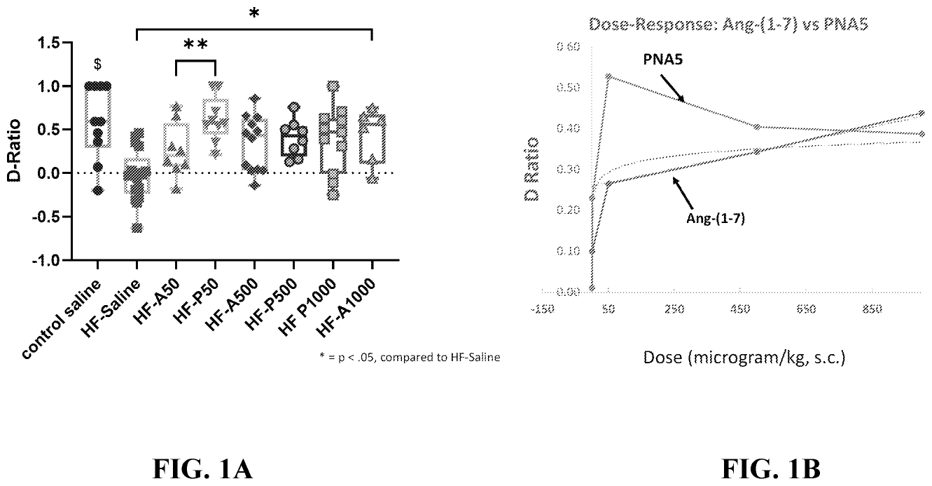

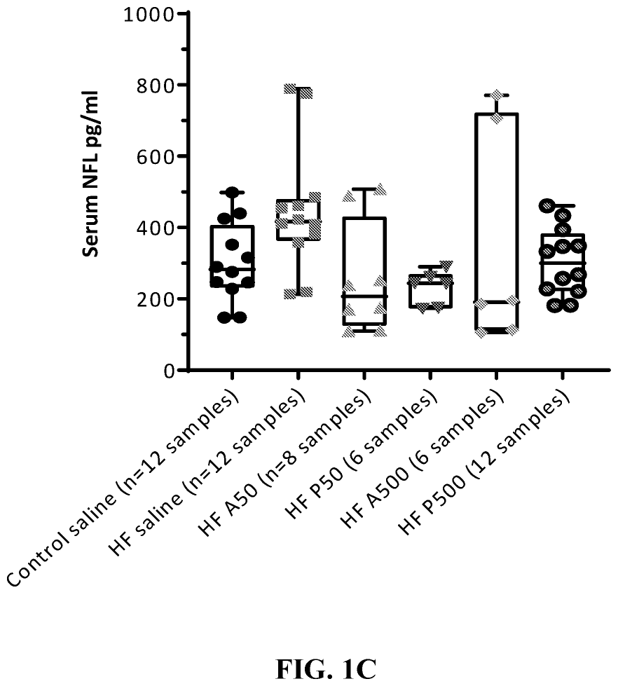

Mas Receptor Agonists Reverse VCI: The results of this study confirm previous findings that the present HF model results in a significant cognitive impairments that model VCI, and that these cognitive impairments may be reversed by Mas receptor agonist therapy. FIGS. 1A and 1B provides the D-ratio measured in the NOR test for all subjects treated with either native Ang-(1-7) or PN-A5. In order to increase power, the 1,000 μg/kg/day Ang-(1-7) and PN-A5 group data are reproduced from an earlier study of subjects treated identically. Confirming our earlier results and previously published studies, the HF-saline group showed a significant impairment in the NOR test relative to controls (p<0.05), and treatment with the Mas receptor agonists native Ang-(1-7) and PN-A5 mitigated or completely reversed that impairment. Furthermore, at the lowest tested dose of 50 μg/kg/day, PN-A5 produced a statistically significant greater improvement relative to the improvement measured for native Ang-(1-7) at the same dose. No statistically significant difference was observed at higher doses suggesting that the therapeutic efficacy is maximized or saturated at the lower end of the dosing range tested in these studies, and further suggesting that PN-A5 has a higher therapeutic efficacy than native Ang-(1-7).

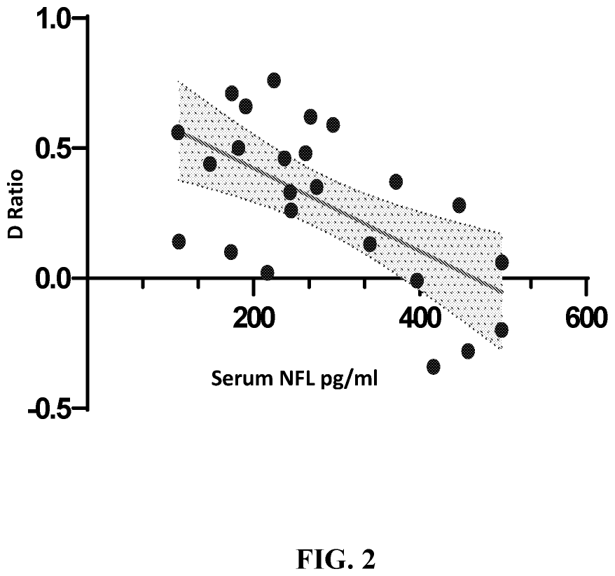

Serum NFL Levels Are A Sensitive Indicator Of HF-Induced Brain Injury: FIG. 1C summarizes the serum NFL levels of the various surgical and treatment groups. As shown, NFL levels were significantly increased in the HF-saline group relative to the Control-saline group (p=0.02). The HF-induced increase in NFL levels was reversed by both native Ang-(1-7) and PN-A5 at both 50 μg/kg/day and 500 μg/kg/day relative to the HF-saline group, and were statistically indistinguishable from controls. Furthermore and as shown in FIG. 2, serum NFL levels were significantly correlated with novel-object recognition performance as measured by the D Ratio (R 2=0.18, p=0.03). These data demonstrate that serum NFL is both a sensitive indicator of VCI and a useful marker to monitor the effectiveness of therapy.

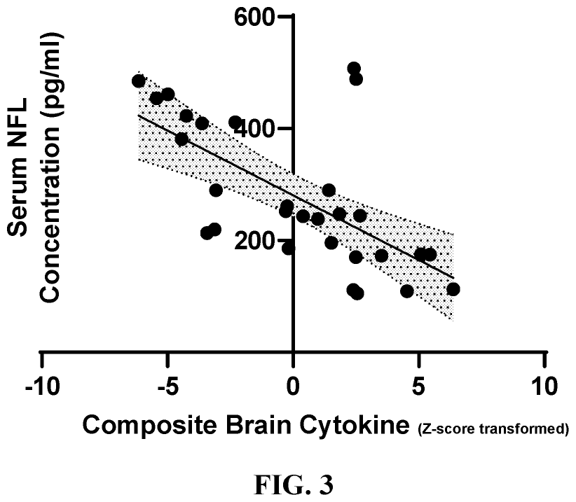

Serum NFL Levels Correlate With Brain Cytokine Levels Following HF-Induced Brain Injury: Brain tissue samples also were obtained at the time of sacrifice. Brain tissue was flash-frozen and the composite brain cytokine levels were subsequently determined. Brain cytokine levels were measured using a MILLIPLEX MAP Mouse High Sensitivity Multiplex immunoassay (MCYTOMAG-70K, Millipore Sigma, Burmingham, Mass) for quantifying inflammatory markers. This assay allows for the simultaneous measurement of the following cytokines: G-CSF, GM-CSF, IFNγ, IL-1a, IL-1B, IL-2, IL-4, IL-5, IL-6, IL-7, IL-9, IL-10, IL-12 (p40), IL-12 (p70), IL-13, IL-15, IL-17, IP-10, CXCL1 (KC), CCL2 (MCP-1), CCL3 (MIP1a), CCL4 (MIP1b), CXCL2 (MIP2), CCL5 (RANTES), TNFα. Differences between groups were analyzed by examined using a Students-T test. All statistical tests and p-values were calculated using MS Excel with Daniel's XL toolbox and alpha was set at the 0.05 level. Error bars represent SEM. Differences among groups (e.g. differences in mean levels over time, differences in change scores from baseline) are assessed using contrasts in cell means in the repeated measures ANOVA. The significance level is set to 0.05. As shown in FIG. 3, serum NFL levels were significantly correlated with the composite brain cytokine score (R 2=0.41, p=0.0002). These data demonstrate that serum NFL is a sensitive surrogate marker for disease-relevant biochemical changes within the CNS.

### Example 2: Native Ang-(1-7) Reverses CABG-Induced Increase in Human Serum NFL Levels

It is well documented that patients undergoing coronary artery bypass grafting (CABG) tend to experience post-surgical cognitive dysfunction. The present study elucidates the response in serum NFL levels to CABG and how that response may be modulated by Ang-(1-7) treatment. Potential CABG participants underwent a screening visit between 2 and 15 days prior to CABG surgery, including a full medical history and physical examination to rule out dementia (Mini-Mental State Exam score less than 25), and depression (Geriatric Depression Scale score greater than 10). Between 1 and 15 days prior to CABG surgery, participants underwent neuropsychological evaluation including memory testing and blood draws for clinical pathology assessment, NFL biomarker assessment. Administration of the first dose Ang-(1-7) or placebo began approximately two hours prior to CABG surgery and continued once a day for an additional 20 days. At 21 days post-surgery, additional blood samples were obtained, and serum NFL levels were measured.

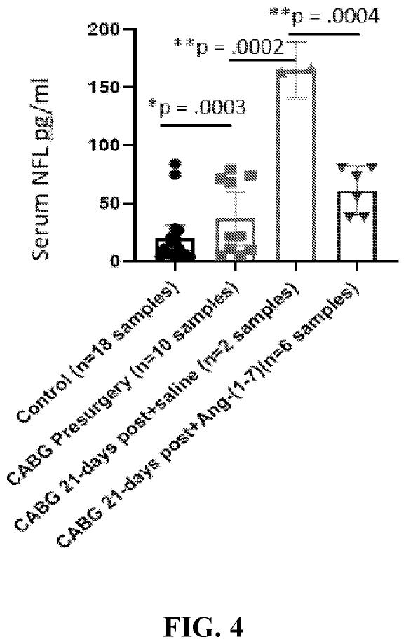

The serum NFL concentration was measure in six subjects prior to CABG surgery and in 18 age-matched control subjects. Following CABG, the six subjects were administered 200 μg/kg/day of native Ang-(1-7) by subcutaneous injection for 21 days. Two subjects were administered saline vehicle control. As shown in FIG. 4, presurgical CABG patients had a statistically significantly increased serum NFL concentration (p=0.0003) relative to healthy controls. Following surgery, serum NFL levels increased about four-fold in the patients treated with saline vehicle compared to pre-surgery level (p=0.0002). However, Ang-(1-7) treatment significantly reduced that post-surgical increase. Taken together with the mechanistic information obtained from the murine VCI model described above, these data indicate that native Ang-(1-7), a Mas receptor agonist, is protective against post-surgical cognitive dysfunction specifically. These data also further validate serum NFL as a sensitive biomarker for this disease process and treatment efficacy.

### Example 3: Serum NFL Levels Correlate with VCI Diagnosis in Human Subjects

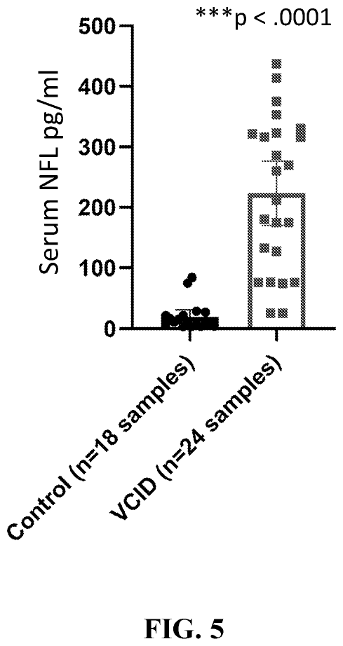

Post-mortem human samples were used to evaluate serum NFL level as a biomarker of VCI. Serum samples were obtained from the Banner Alzheimer's Biobank (Phoenix, AZ) for 24 subjects diagnosed as having VCI and 18 age-matched controls. Serum NFL levels were determined using the Quanterix Simoa assay according to the manufacturer's protocol. As shown in FIG. 5, serum NFL levels were elevated by about 858% in samples obtained from VCI patients relative to age-matched controls (p<0.0001).

Post-mortem human samples were used to evaluate serum NFL level as a biomarker of VCI. Serum samples were obtained from the Banner Alzheimer's Biobank (Phoenix, AZ) for 24 subjects diagnosed as having VCI and 18 age-matched controls. Serum NFL levels were determined using the Quanterix Simoa assay according to the manufacturer's protocol. As shown in FIG. 5, serum NFL levels were elevated by about 858% in samples obtained from VCI patients relative to age-matched controls (p<0.0001).

### Example 4

This example characterizes neurofilament light (NfL) as a prognostic biomarker for treatment of vascular contributions to cognitive impairment and dementia.

Decreased cerebral blood flow and systemic inflammation during heart failure (HF) increase the risk for vascular contributions to cognitive impairment and dementia (VCID) and Alzheimer disease-related dementias (ADRD). Previous experiments demonstrated that PNA5, a novel glycosylated angiotensin 1-7 (Ang-(1-7)) Mas receptor (MasR) agonist peptide, is an effective therapy to rescue cognitive impairment in our preclinical model of VCID. Neurofilament light (NfL) protein concentration is correlated with cognitive impairment and elevated in neurodegenerative diseases, hypoxic brain injury, and cardiac disease. A goal of the following experiments was to determine (1) if treatment with Ang-(1-7)/MasR agonists can rescue cognitive impairment and decrease VCID-induced increases in NfL levels as compared to HF-saline treated mice and, (2) if NfL levels correlate with measures of cognitive function and brain cytokines in our VCID model.

VCID was induced in C57BL/6 male mice via myocardial infarction (MI). At 5 weeks post-MI, mice were treated with daily subcutaneous injections for 24 days, 5 weeks after MI, with PNA5 or angiotensin 1-7 (500 microg/kg/day or 50 microg/kg/day) or saline (n=15/group). Following the 24-day treatment protocol, cognitive function was assessed using the Novel Object Recognition (NOR) test. Cardiac function was measured by echocardiography and plasma concentrations of NfL were quantified using a Quanterix Simoa assay. Brain and circulating cytokine levels were determined with a MILLIPLEX MAP Mouse High Sensitivity Multiplex Immunoassay. Treatment groups were compared via ANOVA, significance was set at p<0.05.

Treatment with Ang-(1-7)/MasR agonists reversed VCID-induced cognitive impairment and significantly decreased NfL levels in our mouse model of VCID as compared to HF-saline treated mice. Further, NfL levels were significantly negatively correlated with cognitive scores and the concentrations of multiple pleiotropic cytokines in the brain.

These data indicate that treatment with Ang-(1-7)/MasR agonists rescues cognitive impairment and decreases plasma NfL relative to HF-saline-treated animals in our VCID mouse model. Further, levels of NfL are significantly negatively correlated with cognitive function and with several brain cytokine concentrations.

### Background

Increases in brain and systemic inflammation, and decreased brain blood flow, as seen during heart failure (HF) (see, Qiu C, et al., Arch Intern Med. 2006; 166(9):1003-8), are strongly correlated with the development of vascular contributions to cognitive impairment and dementia (VCID) and Alzheimer's disease-related dementias (ADRD) (see, Audia C, et al. Pathophysiologic relationship between Alzheimer's disease, cerebrovascular disease, and cardiovascular risk: a review and synthesis. Alzheimers Dement. 2017; Corriveau R A, et al., Cell Mol Neurobiol. 2016; 36:281-8). With an aging population and a strong link between cardiovascular and cerebrovascular disease and subsequent cognitive impairment and dementia, there is a growing need for safe and effective therapies to protect cognitive function in individuals at risk for VCID (see, Mp M, Ra C, Dm W. Biochim Biophys Acta. 2016; 1862(5):857-9).

Angiotensin 1-7 (Ang-(1-7)) is a clinically safe peptide (see, Rodgers K E, Oliver J, DiZerega G S. Cancer Chemother Pharmacol. 2006; 57(5):559-68) that decreases inflammation and increases cerebral blood flow in our VCID preclinical model (see, Hay M, et al., Behav Neurosci. 2017; 131(1):99-114; Hay M, et al., J Pharmacol Exp Ther. 2019; 369:9-25; Jiang T, et al., Br J Pharmacol. 2014; 171(18):4222-32). Through targeting Mas receptors (MasR), located on neurons, microglia, and vascular endothelial cells in the brain (see, Ferrario C M. Angiotensin-converting enzyme 2 and angiotensin-(1-7): an evolving story in cardiovascular regulation. Hypertension. 2006), Ang-(1-7) decreases brain inflammation and increases cerebral circulation (see, Ferrario C M. Angiotensin-converting enzyme 2 and angiotensin-(1-7): an evolving story in cardiovascular regulation. Hypertension. 2006). MasRs that regulate vasodilation are highly expressed in the hippocampus and perirhinal cortex, which makes them an ideal target for treating brain disease related to hypoxia and inflammation that impair memory, such as VCID and ADRD (see, Hay M, et al., Behav Neurosci. 2017; 131(1):99-114; Hay M, et al., J Pharmacol Exp Ther. 2019; 369:9-25). Our (the inventors) collaborative team has developed and optimized a novel, synthetic Ang-(1-7)-derived glycopeptide, PNA5, that selectively activates MasR and has improved bioavailability and brain penetration compared to the native Ang-(1-7) peptide. We have previously shown that PNA5 targets MasR, reverses cognitive impairment in our preclinical VCID model, and inhibits circulating and brain inflammatory cytokine production (see, Hay M, et al., J Pharmacol Exp Ther. 2019; 369:9-25).

Neurofilament light protein (NfL) is an intermediate filament protein that is a component of the cytoskeleton of neurons and is abundantly expressed in axons (see, Zetterberg H. Neurofilament light: a dynamic cross-disease fluid biomarker for neurodegeneration. Neuron. 2016; Ashton N J, et al., Acta Neuropathol Commun. 2019; 7(1):5). NfL levels are increased in the brain following axonal damage and neurodegeneration (see, Zetterberg H. Neurofilament light: a dynamic cross-disease fluid biomarker for neurodegeneration. Neuron. 2016; Ashton N J, et al., Acta Neuropathol Commun. 2019; 7(1):5) and increases in serum NfL correlate with cerebrospinal fluid (CSF) levels and increased cognitive impairment (see, Gaetani L, et al., J Neurol. 2019; 266(9):2157-63). NfL has been found to be elevated in subjects suffering from multiple sclerosis (see, Gaetani L, et al., J Neurol. 2019; 266(9):2157-63; Villar L M, et al., Eur J Neurol. 2015; 22(8):1169-75), as well as traumatic brain injury (see, Thelin E, et al., J Neurotrauma. 2019; 36(20):2850-62; Shahim P, et al., Neurology. 2020; 95(6):e610-22), hypoxic brain injury (see, Nielsen H H, et al., Front Neurol. 2020; 11:11), and cardiac disease and related surgeries (see, Wiberg S, et al. Associations between mean arterial pressure during cardiopulmonary bypass and biomarkers of cerebral injury in patients undergoing cardiac surgery: secondary results from a randomized controlled trial. Interact Cardiovasc Thorac Surg. 2020).

In the following experiments it was hypothesized that (1) treatment with Ang-(1-7)/MasR agonists will rescue cognitive impairment and decrease VCID-induced NfL levels as compared to HF-saline treated mice, and (2) NfL levels will correlate with measures of both cognitive function and VCID-induced increases in brain cytokines. Our (the inventors) results indicate that circulating NfL serves as a prognostic biomarker of cognitive impairment in VCID. Further, the results indicate NfL as a candidate biomarker to identify target engagement following treatment with Ang-(1-7)/MasR agonists in VCID.

### Materials and Methods

### Animal

Three-month-old (— 25-30 g) C57BL/6 male mice (Jackson Laboratories, Bar Harbor, Maine) were ordered and housed 3 per cage in a temperature and humidity-controlled facility on a 12 h light/dark cycle. Mice are acclimated upon receival for a week before undergoing MI. Water and chow were available ab libitum for the duration of the experiments. Experiments were performed with adherence to guidelines approved by the Institutional Animal Care and Use Committee at the University of Arizona and in accordance with the National Institutes of Health Guidelines for the Care and Use of Laboratory Animals. The number of animals needed per experimental group was determined using a G*Power analysis.

### VCID Animal Model

Mice were weighed prior to surgery and anesthetized with 2.5% isoflurane in a mixture of air and O₂. Left coronary artery (LCA) ligation was used to induce myocardial infarction (MI) using the procedure described in Gao et al. [18]. In short, a left-sided thoracotomy was performed at the fourth intercostal space and the LCA was sutured to induce a permanent ligation and MI. Occlusion of the LCA was confirmed by observing myocardial blanching of the left ventricular anterior wall. Control mice underwent sham surgeries, undergoing the same procedure except for the permanent LCA ligation.

### Experimental Protocol

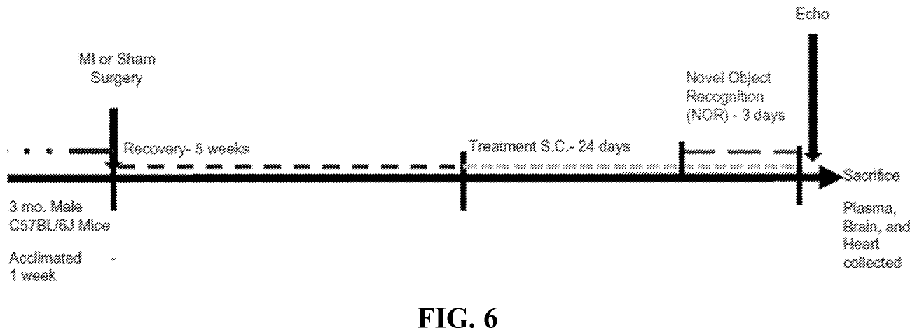

Mice were assigned randomly to the following treatment groups: Control-Saline (sham MI surgery, treated with saline), HF-treated (HF-PNA5, or HF-Ang-(1-7) with either 50 μg/kg/day, or 500 μg/kg/day), or HF-saline. Mice recovered for 5 weeks following MI before receiving 24 days of daily subcutaneous drug treatment. Within the last 3 days of injections, animals underwent Novel Object Recognition (NOR) testing (FIG. 6). Animal treatment groups were as listed: heart failure (HF)-saline treated n=15, (HF-Saline), Sham surgery-saline treated n=15 (Control-Saline), HF-Ang-(1-7) at 500 μg/kilogram body weight/day for 24 days (HF-A500) n=15, HF-Ang-(1-7) at 50 μg/kg body weight/day for 24 days (HF-A50) n=15, HF-PNA5 at 500 μg/kilogram body weight/day (HF-P500) n=15, HF-PNA5 at 50 μg/kilogram body weight/day (HF-P50) n=15. Following MI, up to 7 mice died within a group resulting in varying number of mice per group that were available to undergo treatment, NOR testing, and provide serum samples.

### Echocardiography

Transthoracic, high-resolution ultrasound was performed the day of, or up to 2 days prior to, tissue harvesting using a Vevo 2100 High-Resolution Imaging System (Visual Sonics, Toronto, ON, Canada) and a 25-MHz transducer. Mouse chest hair was removed using a chemical depilatory. Echocardiographs were performed on anesthetized mice under 2% isoflurane and an 02/air mixture. Echocardiographic images were taken in B mode from the parasternal angle both on long and short-axis view at three levels starting at the apex, mid-ventricle, and top of the ventricle. The thickness of the left ventricular wall, cardiac chamber dimensions, interventricular septum, left ventricular posterior wall thickness, and left ventricular internal dimension were measured using Vevo 2100® analytic software (Visual Sonics, Toronto, ON, Canada). Only MI animals showing evidence of a significant MI, defined by significant wall motion abnormalities, and reduced contractile function, were included in the study.

### Preparation of Ang-(1-7) and the Glycosylated Ang1-7, PNA5 Peptides

To prepare the injections, solid peptides were dissolved in double distilled water and stored in a −20° C. freezer in aliquots of 1.5 mL at a stock concentration of 1 mg/mL. The stock drugs were diluted daily in sterile saline to make the following treatment concentrations: A50 50 μg/kg (1.5 mg Ang-(1-7)/300 mL saline), P50 50 μg/kg (1.5 mg PNA5/300 mL saline), A500 500 μg/kg (15 mg Ang-(1-7)/300 mL saline), P500 500 μg/kg (15 mg PNA5/300 mL saline). Treatments were stored in a 4° C. refrigerator in prefilled syringes (peptide solutions are stable up to 28 days when stored at 4° C.).

### Subcutaneous Injections

Daily treatment was delivered subcutaneously either at the scruff or inguinal area for 24 days using a 15-gauge needle. All prepared drugs were kept in the 4° C. refrigerator before use and transported on ice before injection. All procedures were performed within the housing facility to minimize stress.

### Novel Object Recognition (NOR) Test

Mouse cognition was evaluated using the Novel Object Recognition test. For this test, the arena was an evenly illuminated chamber (12×12×12 cm) placed in a light and sound-controlled room. The walls of the arena were white, and the floor had a grid to ensure that the placement of the objects remained the same between phases of the test. Exploratory behavior was measured using a digital camera and evaluated by calculating the time spent exploring each object. Orientation of the nose toward an object at a distance of ˜2 cm or less was considered exploratory behavior (see, Ennaceur A, et al., Behav Brain Res. 1988; 31(1):47-59). Other behaviors including rearing on the object and resting against the object were not considered exploratory. Importantly, we have demonstrated that exploration is similar in both Control-Saline and HF-Saline treated mice, indicating that MI does not have a significant impact on mouse movement after 8 weeks of HF (see, Hay M, et al., Behav Neurosci. 2017; 131(1):99-114; Hay M, et al., J Pharmacol Exp Ther. 2019; 369:9-25). Individuals scoring mouse exploration were blinded to treatment group. Three sets of distinct objects were used, variable in size, color, and shape, and made of either plastic, glass, or wood.

For the habituation phase of the test, mice were habituated to an empty testing arena for 10 min each day for 2 days. For the learning phase of the test, two identical objects were placed in the testing arena and the mouse was allowed to explore the objects for 6 min. After the 6 min of exploration, mice were returned to their home cage for 2 h. For the memory testing phase, one of the objects was replaced with a novel object and the mice were returned to the testing arena and allowed to explore the objects for 2 min. In between each phase, the testing boxes were cleaned with 70% ETOH to prevent the influence of olfactory cues. Analysis: Recognition memory was scored using a discriminatory ratio (discrimination ratio) calculated by: discrimination ratio=the time spent exploring the novel object (tₙₒᵥₑₗ) minus the time spent exploring the familiar object (t_{familiar}) divided by the total exploration time:

Discrimination ratio=(tₙₒᵥₑₗ−t_{familiar})/(total exploration time). (1)

A positive discrimination ratio score indicates that the mouse spent more time at the novel object than the familiar object, while a negative score indicates that the mouse spent more time with the familiar object than the novel object. A zero-discrimination ratio score indicates a null preference. Typically, in healthy states, mice will spend a greater amount of time with the novel object than the familiar object. Discrimination ratios between groups were analyzed using ANOVA, followed by Dunnett's post with significance set at p<0.05.

### Measurement of Neurofilament Light (NfL)

Upon killing, blood was collected via heparinized syringe from the neck following decapitation. Collected blood was centrifuged and the plasma stored at −80° C. before shipment to PBL Assay Science for measurement of NfL using a Quanterix-Simoa assay (NF-LIGHTED 103186, PBL, Neurofilament-Light Advantage Assay Kit).

### Brain Lysis

Mice were anesthetized with isoflurane and rapidly decapitated. Brains were separated from the skull and quickly cut into two hemispheres. The halves of the brain were then rapidly frozen in corning tubes in liquid nitrogen and stored at −80° C. One half of each brain was lysed using 1 mL RIPA buffer (1% Triton X-100, 0.1% SDS, 1×PBS) with protease (protease inhibitor cocktail 100× [Sigma P8340]) and phosphatase inhibitors (phosphatase inhibitor cocktail 100× [Sigma P5726]). Tissue was then sonicated using an ultrasonic cell disruptor at 30% 40% amplitude for 1 min. Samples were returned to ice in between sonication to prevent the tissue from warming. Samples were then centrifuged at 4° C. or 15 min at 13,000×g. The supernatant was collected and stored at −80° C. Total protein was measured in the range of 5-mg/mL. Brain lysates were used for multiplex immunoassays.

### Cytokine Measurement

Cytokine concentrations were measured in the brain lysates and plasma samples by multiplex immunoassay using a MAGPIX Multiplexing Instrument and accompanying Multiplex Analyst software (MILLIPLEX MAP Mouse Cytokine/Chemokine Magnetic Bead Panel-Immunology Multiplex Assay; MCYTOMAG-70 K; Millipore Sigma, Burlington, Massachusetts). Simultaneous measurements of granulocyte colony-stimulating factor (GCSF), granulocyte-macrophage colony-stimulating factor, interferon, interleukin (IL)-1a, IL-1B, IL-2, IL-4, IL-5, IL-6, IL-7, IL-9, IL-10, IL-12 (p40), IL-12 (p70), IL-13, IL-15, IL-17, interferon-induced protein 10, chemokine (C-X-C motif) (CXC) ligand 1 (KC), chemokine (C-C motif) ligand (CCL) 2 (monocyte chemoattractant protein-1), CCL3 [macrophage inflammatory protein (MIP) 1a], CCL4 (MIP1b), CXC ligand 2 (MIP2), CCL5 [RANTES (regulated on activation normal T cell expressed and secreted)], and tumor necrosis factor-alpha (TNF-α) were captured. All samples were run in duplicate, and the results were analyzed using ANOVA and relevant post using GraphPad Prism9. To create a composite cytokine score, we calculated the z-scores for IL-2, IL-13, and IL-17 brain cytokines. Z-scores were then added to create a composite cytokine score. The z-score was calculated for each cytokine using the following equation:

z score=(individual value−mean value)/SD. (2)

### Heart Tissue Fixation

Mice were anesthetized with isoflurane and rapidly decapitated. The heart was exposed by opening the chest cavity and excised. The heart, while still beating, was then washed in ice cold PBS, dried, and weighed. Hearts were then cut longitudinally, exposing the interventricular septum and both ventricle chambers, and immersed in 10% formalin overnight at 4° C. Hearts were then dehydrated in methanol with at least two methanol changes before being cleared with xylene for 45 min. Hearts were then wax permeabilized and paraffin embedded.

### Histochemistry

Paraffin-embedded hearts were sectioned on a microtome to a thickness of 5-microns and mounted onto polarized slides. They were then stained with hematoxylin and eosin to visualize heart morphology and cellular infiltrates.

### Statistical Analysis

Data were analyzed using GraphPad Prism 9.0. Values are expressed as mean±SE. The Shapiro-Wilk W-test was used for determining normal distribution of the data. Differences between multiple groups were analyzed with ANOVA followed by appropriate post hoc group comparisons where indicated. Comparison of NfL levels between the Control-Saline and HF-Saline groups were analyzed with an unpaired t-test. Statistical significance was set at p value <0.05. Association between NfL levels and discrimination ratio scores and the brain cytokines were analyzed using Pearson correlation. Statistical significance was set at p value <0.05. The discrimination ratio vs NfL linear regression and brain cytokine vs NfL linear regression was used to generate a best-fit line.

### Results

### HF in VCID Model is Confirmed Through Loss of Ventricular Function

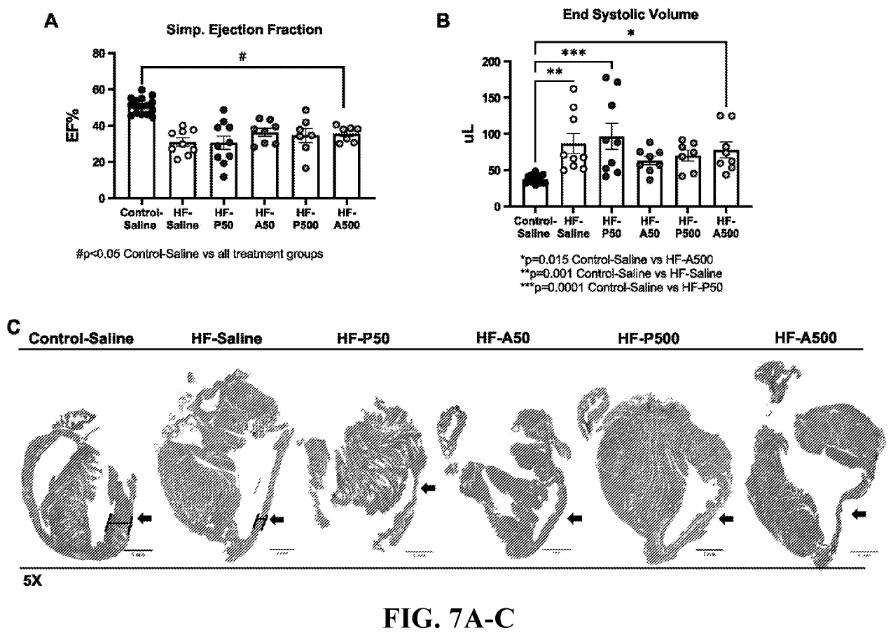

Our (the inventors) previous studies demonstrated that there is a significant deficit in cardiac function by 4 weeks post-MI (see, Hay M, et al., Behav Neurosci. 2017; 131(1):99-114; Hay M, et al., J Pharmacol Exp Ther. 2019; 369:9-25). Accordingly, echocardiography was performed at 8 weeks post-MI using the Simpson's method, the most used approach in clinical practice. As illustrated in FIG. 7A, at 8 weeks post-MI, ejection fractions (EF) were decreased in HF-Saline and Ang-(1-7) and PNA5-treated mice compared to Control-Saline-treated mice (Control-Saline, mean 50.98, ±SE 1.12 n=17 vs HF-Saline, mean 30.99±SE 2.26, n=9, HF-A500, mean 35.56±SE 1.60, n=7, HF-P500, 34.54±SE 3.86, n=7, HF-P50, mean, 30.57±SE 3.66, n=10, HF-A50 36.30±SE 2.18, n=8; respectively, p≤0.0002 ANOVA, Dunnett's post). Further, at 8 week post-MI, end systolic volume (ESV) were significantly increased in all groups HF-Saline, as compared to Control-Saline treated mice (Control-Saline, mean 37.78±SE 1.30 n=17 vs HF-VCID, mean 86.97±SE 13.90, n=9, p=0.0013, HF-A500, mean 78.29±SE 11.02, n=8, p=0.0149, HF-P50, 96.79±SE 17.87, n=9, p=0.0001; ANOVA, Dunnett's post) (FIG. 7B). As observed in dilated hearts, our VCID mouse model has a thinned left ventricular wall (see, Rumberger J A. Ventricular dilatation and remodeling after myocardial infarction. Mayo Clinic Proc. 1994). FIG. 7C is an illustrative example of left ventricular wall thinning in HF-Saline, PNA5, and Ang-(1-7) treatment treated mice compared to Control-Saline treated mice (indicated by black arrow).

### Ang-(1-7)/MasR Activation Rescues Cognitive Impairment in VCID Mice

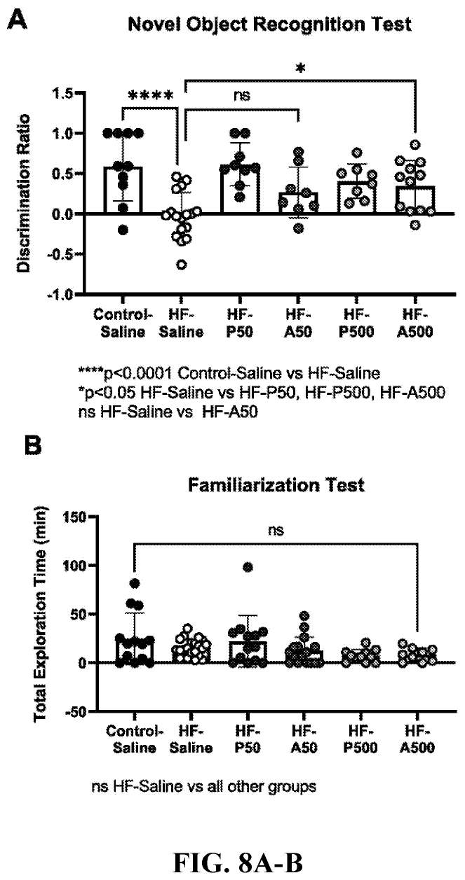

As illustrated in FIG. 8A, treatment with Ang-(1-7)/MasR agonists rescues cognitive impairment in our mouse model of VCID. Cognitive function was measured using the NOR test following 24 days of treatment with either 50 or 500 μg/kg/day of either Ang-(1-7) or PNA5. Cognitive function was quantified using the Discrimination ratio score, with a negative, or zero Discrimination ratio indicating impaired cognitive function. HF mice treated with saline had an average negative Discrimination ratio of −0.026±SE 0.07 that was significantly lower than Control-Saline treated mice (mean 0.590±SE 0.13, p<0.001, ANOVA, Dunnett's post). This indicates, as previously described, that in our model of VCID, HF results in a decrease in cognitive function (see, Hay M, et al., Behav Neurosci. 2017; 131(1):99-114; Hay M, et al., J Pharmacol Exp Ther. 2019; 369:9-25).

Compared to HF-Saline animals, both treatment with PNA5 and Ang-(1-7) rescued the cognitive function in the VCID model. VCID mice treated with PNA5 at 50 or 500 μg/kg/day resulted in a discrimination ratio of 0.61±SE 0.08, n=9, and 0.40±SE 0.08, n=8, respectively (HF-P50 p<0.0001, HF-P500, p=0.01; ANOVA, Dunnett's post). Likewise, treatment with Ang-(1-7) at 500 μg/kg/day also rescued HF-induced cognitive impairment mice (HF-A500, mean 0.34±SE 0.09, n=12, p=0.013; ANOVA, Dunnett's post).

We have previously demonstrated that VCID mice do not demonstrate altered levels of activity or anxiety compared to control mice during the NOR test (see, Hay M, et al., Behav Neurosci. 2017; 131(1):99-114; Hay M, et al., J Pharmacol Exp Ther. 2019; 369:9-25). Consistent with our previous publications, FIG. 8B demonstrates that our mouse model of VCID shows no significant difference in total exploration time during the familiarization phase in all groups. These results indicate that in comparison to Control-Saline-treated mice, VCID mice do not demonstrate altered levels of activity or anxiety (Control-Saline, mean 24.80±SE 7.32 n=13, HF-Saline, mean 15.61±SE 1.91, n=21, HF-P50, mean 22.32±SE 7.34, n=13, HF-A50, 12.26±SE 3.59, n=16, HF-P500, mean, 7.36±SE 1.93, n=11, HF-A500 8.47±SE 2.34, n=9; respectively, p >0.05, not significant ANOVA).

### NfL Plasma Levels in VCID Mice and Effects of Ang-(1-7)/Mas Receptor Activation

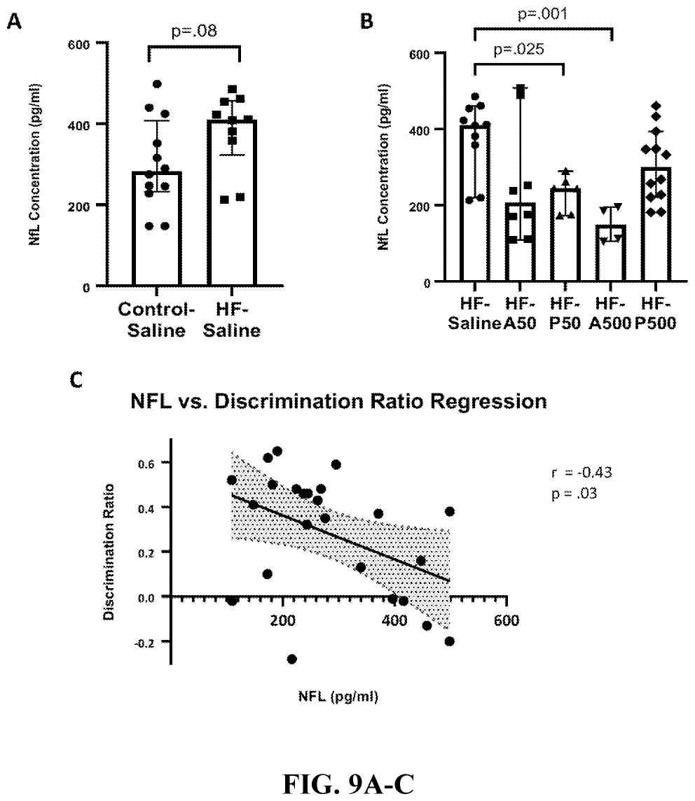

Levels of NfL were higher in the HF-Saline-treated mice relative to levels in the Control-Saline mice. The mean NfL level in the HF-VCID mice was 381.6±SE 30.0, (n=10) as compared to Control-Saline-treated mice at 301.0±SE 32.0, (n=12). Student's t-test resulted in p=0.08 and did not reach significance (FIG. 9A).

As illustrated in FIG. 9B, NfL concentrations were significantly lower following treatment with either A50, P50, or A500 as compared to HF-Saline-treated mice (HF-saline, mean 381.6±SE 32.0, (n=12), HF-A50, mean 256.7±SE 55.8, (n=8), p=0.025; HF-P50 mean 231.1±SE 19.3, (n=6), p=0.025; HF-A500 mean 149.8±SE 23.6, (n=4), p=0.001; Kruskal-Wallis ANOVA, Dunn's post).

To assess the relationship between cognitive impairment observed in our VCID model and circulating NfL, NfL levels were correlated to the discrimination ratio score of all mouse groups (FIG. 9C). Plasma NfL was significantly negatively correlated with the discrimination ratio score (Pearson's r=−0.43, p=0.03), demonstrating that cognitive abilities decrease in this model as neuronal injury markers increase.

Treatment with Ang1-7/MasR Agonists Affects Levels of Both Brain and Circulating Cytokines

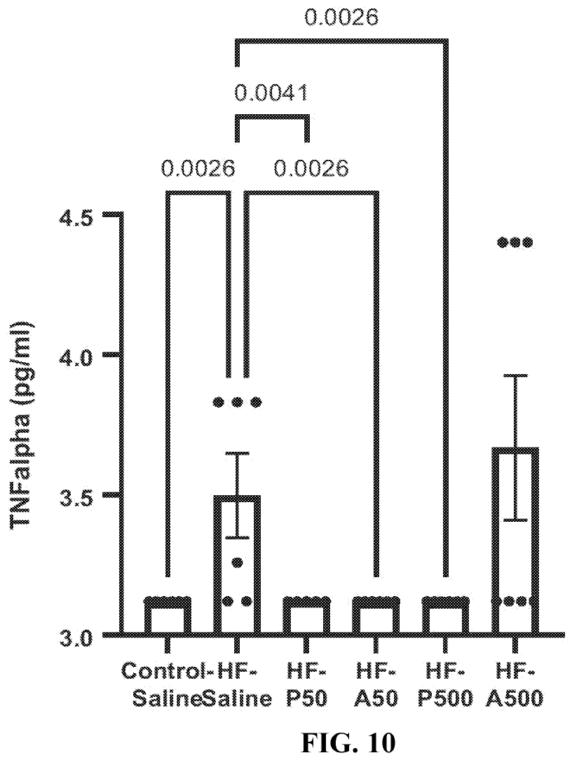

Using the MILLIPLEX MAP Mouse High Sensitivity Multiplex Immunoassay, we measured plasma and brain inflammatory profiles after PNA5 and Ang-(1-7) treatments at both the 50 and 500 μg/kilograms/day doses. Among 25 cytokines measured in the plasma, only TNFα exhibited a significant difference between HF-Saline and Control-Saline-treated mice (3.50±SE 0.15, n=6 vs means 3.12±SE 0.01, n=6, respectively, p=0.003; ANOVA, Dunnett's post hoc). PNA5 and Ang-(1-7) inhibited this HF-induced increase in TNFα as compared to HF-saline groups (HF-P50 mean 3.12 pg/mL±SE 0.00, n=5 p=0.004; HF-A50 mean 3.12 pg/mL±SE 0.00, n=6, p=0.003; HF-P500 mean 3.12 pg/mL±SE 0.00, n=6, p=0.003, ANOVA, Dunnett's post; values that were at or below assay threshold of 3.12 pg/mL were represented as 3.12 pg/mL) (FIG. 10).

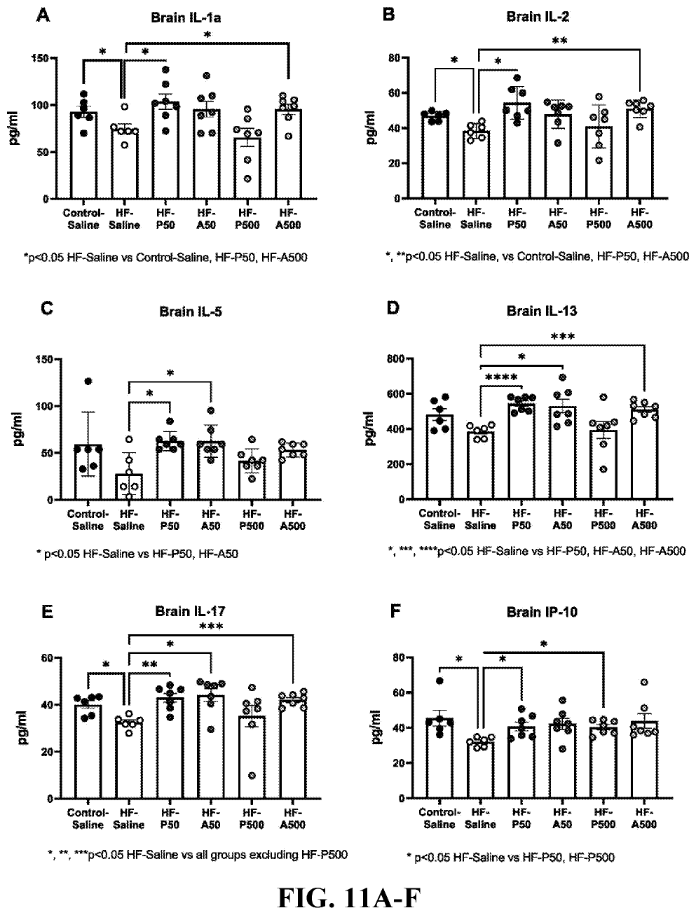

Cytokine levels from brain lysates are summarized in Table 2 and illustrated in FIG. 11. Of the 25 cytokines tested treatments with P50 significantly increased levels of putative-neuroprotective cytokines IL-1α, IL-2, IL-5, IL-13, IL-17, and IP-10, compared to HF-Saline treated mice, ANOVA, Dunnett's post hoc, p<0.05 (FIG. 10A-F). Similarly, treatments with A50 or A500 also significantly increased levels of IL-1α, IL-2, IL-5, IL-13, and IL-17 in comparison to HF-Saline-treated mice, ANOVA, Dunnett's post, p<0.05. Lastly, IL-1α, IL-2, IL-17, and IP-10 were significantly decreased in HF-Saline-treated mice in comparison to Control-Saline treated mice (Table 2).

TABLE 2
IL-1α, IL-2, IL-5, IL-13, IL-17, and IP-10, mean, standard error and number of samples are provided for the following
treatment groups: Control-Saline, HF-Saline, HF-A50 (Ang-(1-7), Ang-(1-7)50 ug/kg), HF-P50 (PNA5 50 ug/kg), HF-
A500 (Ang-(1-7)500 ug/kg), and HF-P500 (PNA5 500 ug/kg). Significant difference between treatment groups and HF-
Saline are provided, *Indicates significance, p < 0.05, (ANOVA, Dunnett's post hoc), ns not significant).

| | Cytokine and chemokine, relative expression (pg/ml) | | | | | | p values | | | | |
|---|---|---|---|---|---|---|---|---|---|---|---|
| | Control | | | | | | Control- | HF-Saline | HF-Saline | HF-Saline | HF-Saline |
| | Saline | HF-Saline | [symbol] | [symbol] | [symbol] | [symbol] | Saline vs. | vs. | vs. | vs. | vs |
| Cytokines | (n = 5-6) | (n = 6) | (n = 6-7) | (n = 6-7) | (n = 7) | (n = 7) | HF-Saline | [symbol] | [symbol] | [symbol] | [symbol] |
| IL-1α | [symbol] | 74.7 ± 5.4 | 95.6 ± 8.4 | 103.7 ± 8.1 | 95.6 ± 5.5 | 65.4 ± 9.7 | 0.048* | 0.027* | ns | ns | 0.040* |
| IL-2 | 46.8 ± 1.1 | [symbol] | [symbol] | 54.3 ± 3.5 | 50.9 ± 1.9 | 40.9 ± 4.6 | 0.015* | 0.013* | ns | ns | 0.002* |
| IL-5 | 59.5 ± 14.0 | 28.0 ± 9.2 | [symbol] | [symbol] | 53.2 ± 2.7 | 41.7 ± 4.9 | ns | 0.020* | 0.026* | ns | ns |
| IL-13 | 481.1 ± 33.4 | 385.9 ± 14.8 | [symbol] | [symbol] | [symbol] | [symbol] | ns | <0.0001* | 0.036* | ns | 0.001* |
| IL-17 | 40.0 ± 1.7 | 32.4 ± 1.1 | 44.1 ± 2.8 | [symbol] | 42.0 ± 1.1 | 35.2 ± 4.6 | 0.021* | 0.004* | 0.020* | ns | 0.0003* |
| IL-10 | 45.5 ± 4.4 | 32.1 ± 1.1 | 42.2 ± 3.2 | 40.8 ± 2.5 | 43.8 ± 4.2 | 40.4 ± 1.5 | 0.049* | 0.023* | ns | 0.005* | ns |

IL-Iα, IL-2, IL-5, IL-13, IL-17, and IP-10, mean, standard error and number of samples are provided for the following treatment groups: Control-Saline, HF-Saline, HF-A50 (Ang-(1-7), Ang-(1-7)50 ug/kg), HF-P50 (PNAS 50 ug/kg), HF-A500 (Ang-(1-7)500 ug/kg), and HF-P500 (PNAS 500 ug/kg). Significant difference between treatment groups and HF-Saline are provided,
*Indicates significance, p < 0.05, (ANOVA, Dunnett's post hoc), ns not significant) [symbol]
indicates data missing or illegible when filed

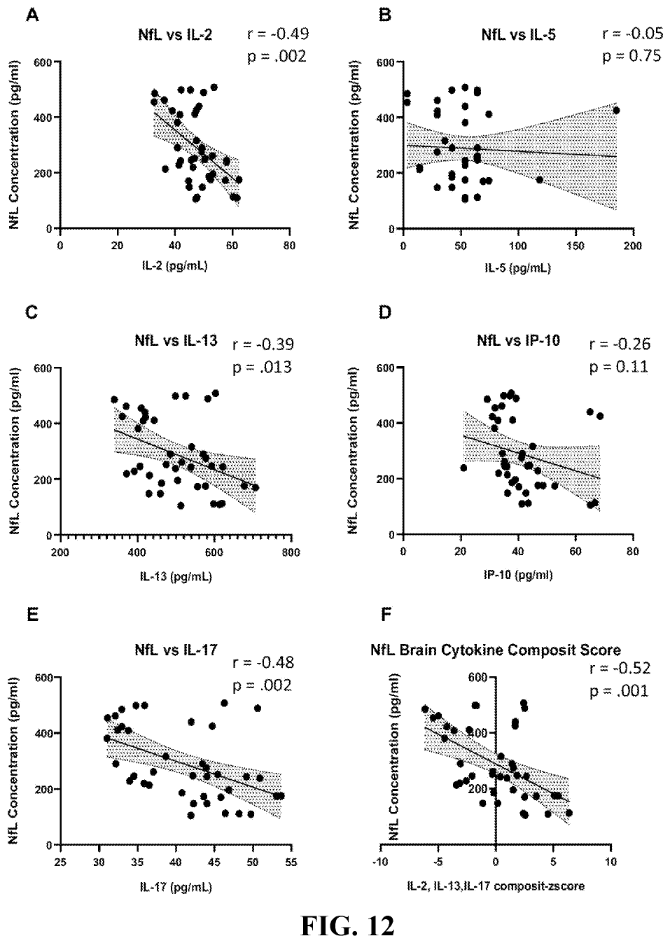

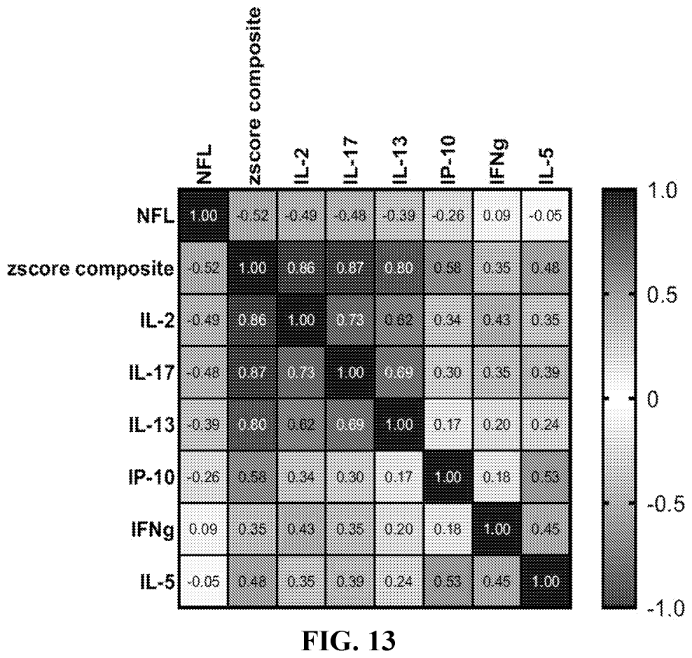

The relationship between levels brain cytokines and NfL was analyzed using Pearson correlation of NfL concentrations and brain cytokines (Table 3, FIG. 12). IL-2, IL-13, IL-17, and the combined cytokine composite score were significantly negatively correlated with NfL plasma concentrations. FIG. 13 is a Pearson's correlation matrix illustrating the correlation coefficients between NfL and those brain cytokines described in Table 3. These data show that (1) NfL is most strongly negatively correlated with the cytokine-composite score and, (2) the levels of IL-2, IL-17 and IL-13 are all strongly positively correlated with each other.

TABLE 3
IL-2, IL-5, IL-13, IL-17, IP-10 and the composite cytokine z-score,
Pearson's correlation r values and affiliated p values following
correlation analysis of NfL levels and cytokine levels in all animal
groups are shown. *Indicates significance, p < 0.05, ns not significant.

| | | | | | | Cytokine |
|---|---|---|---|---|---|---|
| | | | | | | composite |
| Cytokines | IL-2 | IL-5 | IL-13 | IP-10 | IL-17 | z-score |
| r value | [symbol] | −0.05 | −0.39 | −0.26 | −0.48 | −0.52 |
| p value | .002 | 0.74 | .013 | 0.11 | .002 | .001 |

IL-2, IL-5, IL-13, IL-17, IP-10 and the composite cytokine z-score, Pearson's correlation r values and affiliated p values following correlation analysis of NfL levels and cytokine levels in all animal groups are shown.
*Indicates significance, p < 0.05, ns not significant [symbol]
indicates data missing or illegible when filed

### Longitudinal Comparisons of Circulating Inflammatory Cytokine and Chemokines

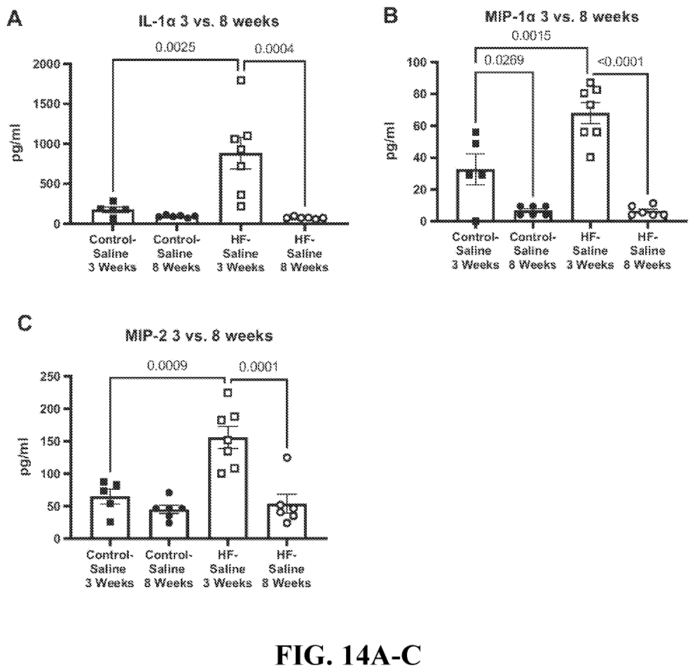

HF is a progressive condition, worsening over time. To highlight the longitudinal impact of HF-induced inflammation, we compared plasma cytokines at 3 weeks post-MI, before the onset of HF, to 8 weeks post-MI, when HF has been established (FIG. 14). A significant decrease in IL-1α, MIP-1α, and MIP-2 plasma concentrations was observed in HF-Saline treated mice between the two time periods (Table 4). Unlike IL-1α, and MIP-2, MIP-1α demonstrated decreased levels 8 weeks post-MI compared to 3 weeks post-MI in both Control-Saline, and HF-Saline treated mice. (IL-1α 3 weeks HF mean 882.3 pg/mL±SE 198.4, n=7 vs 8 week HF mean 209.6 pg/mL±SE 55.7, n=6, p=0.0004; MIP-1α 3 week HF mean 67.9 pg/mL±SE 6.6, n=7 vs 8 week HF mean 6.4 pg/mL±SE 1.3, n=6, p<0.0001; MIP-2 3 weeks HF mean 155.9 pg/mL±SE 17.0, n=7 vs 8 weeks HF mean 54.1 pg/mL±SE 14.7, n=6, p=0.0001; ANOVA, Tukey's post).

TABLE 4
Mean, Standard error, and number of sample values of plasma IL-1α, MIP-1α, and MIP-2 from 3 and
8 weeks post-MI are represented for both treatment groups Control-Saline, and HF-Saline. P values of
the following comparison are provided: 3 weeks post-MI Control-Saline and 3 weeks post-MI HF-Saline,
3 weeks post-MI Control-Saline and 8 weeks post-MI Control-Saline, and 3 weeks post-MI HF-Saline and
8 weeks post-MI HF-Saline. *Indicates significance, p < 0.05, (ANOVA, Tukey's post), ns not significant.

| | | | | | p values | | |
|---|---|---|---|---|---|---|---|
| | Cytokine and chemokine, relative expression | | | | 3 weeks | 3 weeks | |
| | 3 weeks HF | | 8 weeks HF | | Control- | Control- | 3 weeks HF |
| | Control- | 3 weeks HF | Control- | 8 weeks HF | Saline | Saline | Saline |
| | Saline | Hf-Saline | Saline | HF-Saline | vs. 3 weeks | vs. 3 weeks | vs. 8 weeks |
| Cytokines | (n = 5) | (n = 7) | (n = 7) | (n = 6) | HF-Saline | HF-Saline | HF-Saline |
| [symbol] | 176.1 ± 34.8 | 882.3 ± 198.4 | 92.8 ± 5.9 | 74.7 ± 5.4 | 0.003* | 0.964 | 0.0004* |
| [symbol] | [symbol] | [symbol] | [symbol] | 6.4 ± 1.3 | 0.002* | 0.027* | <0.0001* |
| [symbol] | 65.0 ± 11.4 | 155.9 ± 17.0 | 45.2 ± 6.3 | 54.1 ± 14.7 | 0.0009* | 0.764 | 0.0001* |

Mean, Standard error, and number of sample values of plasma [symbol] , and [symbol] from 3 and 8 weeks post- [symbol] are represented for both treatment groups Control-Saline, and HF-Saline. P values of the following comparison are provided: 3 weeks post-MI Control-Saline and 3 weeks post [symbol] Saline, 3 weeks post-MI Control-Saline and 8 weeks post-MI Control-Saline, and 3 weeks post [symbol] Saline and 8 weeks post [symbol] Saline.
*Indicates significance, p < 0.05, (ANOVA, Turkey’s post), ns not significant [symbol]
indicates data missing or illegible when filed

### Conclusions

Vascular contributions to cognitive impairment and dementia (VCID) and Alzheimer's disease-related dementias (ADRD) significantly contribute to the 47 million people world-wide who suffer with dementia. This number is estimated to increase to over 130 million people by 2050. A number of studies have shown that VCID and conversion to ADRD are strongly correlated with vascular disease, inflammation and decreased cerebral brain blood flow (see, Kapasi A, et al., Biochim Biophys Acta Mol Basis Dis. 2016; 1862(5):878-86; Toledo J B, et al., Alzheimer's Dement. 2012; 8(6):483-9; Yarchoan M, et al., Brain. 2012; 135(12):3749-5; van Oijen M, et al., Ann Neurol. 2007; 61(5):403-10; Gorelick P B, et al., Stroke. 2017; 48(10):e284-303; Santos C Y, et al., Alzheimers Dement. 2017; 7(1):69-87; Canobbio I, Abubaker A A, Visconte C, Torti M, Pula G. Role of amyloid peptides in vascular dysfunction and platelet dysregulation in Alzheimer's disease. Front Cell Neurosci. 2015; Janota C, et al., Mol Neurobiol. 2015; 53(6):3793-811). The relationship between vascular disease, cognitive function and progression to dementia and possible AD have been recently reviewed (see, Sweeney M D, et al., Alzheimer's Dement. 2019; 15(1):158-67; Helman A M, et al., Biochim Biophys Acta Mol Basis Dis. 2016; 1862(5):975-82; Rodgers K E, et al., Cancer Chemother Pharmacol. 2006; 57(5):559-68). These authors successfully make the case for a close relationship between cardiovascular risk factors and risk for VCID and ADRD. Furthermore, conversion rates of VCID to dementia has been reported to be within 40-46% within 5 years of diagnosis of VCID (see, Wentzel C, Rockwood K, et al., Neurology. 2001; 57(4):714-6; Hsiung G Y R, et al., Dement Geriatr Cogn Disord. 2006; 22(5-6):413-20). There is an urgent unmet medical need for therapeutics to prevent cognitive decline in individuals at risk for VCID.

The experiments described in Example IV demonstrate that treatment of our (the inventors) VCID mice with Ang-(1-7)/MasR agonists reverse cognitive dysfunction and decrease circulating NfL as compared to HF-saline-treated animals. This change in NfL level is significantly negatively correlated with cognitive function. These early preclinical data suggest that NfL levels might serve as a prognostic to identify changes in cognitive function in VCID. In addition, NfL significantly correlated with treatment induced changes in brain cytokines further suggesting that NfL may potentially also serve as a pharmacodynamic/response biomarker for therapeutic target engagement and disease modification. Extensive studies on levels of NfL in humans with VCID are needed to confirm the utility and reliability of NfL as a biomarker for cognitive impairment in individuals at risk for VCID.

### NIL as a Biomarker in Neurodegenerative Diseases

NfL is released into the CSF and blood upon axonal damage (see, Zetterberg H. Neurofilament light: a dynamic cross-disease fluid biomarker for neurodegeneration. Neuron. 2016). Increases in CSF and blood concentrations of NfL have been found in multiple neurodegenerative diseases (see, Mattsson N, et al., JAMA Neurol. 2017; 74(5):557-66) as well as traumatic brain injury (see, Thelin E, et al., J Neurotrauma. 2019; 36(20):2850-6), hypoxic brain injury (see, Nielsen H H, et al., Front Neurol. 2020; 11:11), and cardiac disease (see, Wiberg S, et al., Interact Cardiovasc Thorac Surg. 2020). Blood NfL levels have been proposed as a biomarker of cognitive decline in Alzheimer's disease (AD) and Parkinson's disease (PD) patients (see, Mattsson N, et al., JAMA Neurol. 2017; 74(5):557-66; Olsson B, et al., Lancet Neurol. 2016; 15(7):673-84). It has been demonstrated that patients experiencing mild cognitive impairment and dementia stages of AD have increased blood and CSF NfL levels (see, Petzold A, et al., Neurodegener Dis. 2007; 4(2-3):185-94), from these results it has been previously proposed that NfL may be able to help predict the progression of AD dementia in patients (see, Mattsson N, et al., JAMA Neurol. 2017; 74(5):557-66; Olsson B, et al., Lancet Neurol. 2016; 15(7):673-84).

Additionally, it has been demonstrated that CSF NfL levels are increased in patients with vascular dementia (see, Rosengren L E, et al., Neurology. 1999; 52(5):1090-3; Skillback T, et al., Neurology. 2014; 83(21):1945-53) and that increased NfL levels are correlated to lower MMSE scores (see, Skillback T, et al., Neurology. 2014; 83(21):1945-53). Further, it has been shown that patients with vascular dementia demonstrate a correlation between increased NfL serum levels and decreased cognitive impairment (see, Ma W, Zhang J, Xu J, Feng D, Wang X, Zhang F. Elevated levels of serum neurofilament light chain associated with cognitive impairment in vascular dementia. Dis Markers. 2020). Studies including CADASIL (a genetic disorder affecting the small blood vessels in the brain), and small vessel disease (SVD) VCID patients also demonstrated increases of NfL correlated to worsened processing speed (see, Cipollini V, Troili F, Giubilei F. Emerging biomarkers in vascular cognitive impairment and dementia: from pathophysiological pathways to clinical application. IJMS. 2019). Experiments described herein provide preclinical support for the hypothesis that NfL may be a prognostic biomarker, as described by the FDA BEST Biomarker Working Group (see, FDA-NIH Biomarker Working Group. BEST (Biomarkers, EndpointS, and other Tools). Silver Spring (MD): Food and Drug Administration (US); 2016-. Available from: https://www.ncbi.nlm.nih.gov/books/NBK326791/Co-published by National Institutes of Health (US), Bethesda (MD)) for the identification of cognitive impairment in individuals at risk for VCID.

Experiments described herein further demonstrate that treatment with Ang-(1-7)/MasR agonists can decrease NfL concentrations in our VCID model as compared to HF-saline treated animals and this decrease in NfL is inversely correlated to several pleiotropic cytokines found in the VCID mouse brain. The correlation of blood NfL levels with neurodegenerative treatment protocols has been demonstrated in previous human studies. In MS patients, studies have shown that blood levels of NfL correlate with MS related disability and can predict the longitudinal course of the disease as well as response to treatment (see, Kuhle J, et al., Neurology. 2019; 92(10):e1007-15; Kapoor R, et al., Neurology. 2020; 95(10):436-44; Varhaug K N, Torkildsen Ø, Myhr K M, Vedeler C A. Neurofilament light chain as a biomarker in multiple sclerosis. Front Neurol. 2019).

### Systemic and Brain Inflammation

A number of different mechanisms have been suggested to contribute to VCID (see, Price B R, et al., J Neurochem. 2018; 144(5):644-50; Price B R, et al., Front Aging Neurosci. 2018; 31:350). Systemic inflammation has been linked to impaired cognitive function (see, Trollor J N, et al., Age (Omaha). 2012; 34(5):1295-308; Bruunsgaard H. A high plasma concentration of TNF-α is associated with dementia in centenarians. J Gerontol Ser A Biol Sci Med Sci. 1999). Specifically circulating TNFα is increased in HF patients (see, Yndestad A, et al., Hear Fail Rev. 2006; 11:83-92; Aukrust P, et al., Am J Cardiol. 1999; 83(3):376-82) and has been demonstrated to contribute to pathologic cognitive changes (see, Bruunsgaard H. A high plasma concentration of TNF-α is associated with dementia in centenarians. J Gerontol Ser A Biol Sci Med Sci. 1999). Experiments described herein observed that TNFα was significantly increased in the HF-Saline treated mice compared to the Control-Saline treated mice, thereby possibly contributing to systemic inflammation in our VCID mouse model. Importantly, treatment with Ang-(1-7)/MasR agonists in VCID mice reversed not only cognitive impairment, but also decreased NfL levels, and systemic TNFα concentrations. These data provide further evidence that NfL serves as a biomarker to indicate target engagement of the disease-modifying treatment with our Ang-(1-7)/MasR agonists.

We (the inventors) have also begun to identify longitudinal changes in circulating inflammatory cytokine profiles in our VCID mouse model. We compared changes in systemic cytokines in animals with 3 weeks of HF to animals with 8 weeks of HF. Previously, we have identified inflammatory biomarkers associated with the early progression of VCID following the onset of HF (3 weeks following MI) (see, Hay M, et al., Behav Neurosci. 2017; 131(1):99-114). IL-1α, MIP-1α, MIP-2, and GCSF in HF-Saline mice were significantly increased compared to the Control-Saline treated mice 3 weeks following MI (see, Hay M, et al., Behav Neurosci. 2017; 131(1):99-114). These data reveal a longitudinal decrease in IL-1α, MIP-1α, and MIP-2 which provides insight into the inflammatory mechanism of VCID development.

Regarding brain cytokines, HF-Saline treated mice had decreased levels of IL-1a IL-2, IL-17, and IP-10 in comparison to Control-Saline treated mice. Treatment with P50 significantly increased IL-1a, IL-2, IL-5, IL-13, IL-17 and IP-10 levels, and treatments with Ang-(1-7) also significantly increased levels of IL-1a, IL-2, IL-5, IL-13, and IL-17 in comparison to HF-Saline treated mice.

IL-1α has been shown to have brain protective qualities in mouse stroke models and promotes neurorepair when mice are treated with delayed administration of IL-1a (see, Salmeron K, et al., J Neuroinflammation. 2019; 16(1):222). Other studies have demonstrated that IL-1a induces angiogenesis in brain endothelial cells in vitro (see, Salmeron K, et al., J Neurochem. 2016; 136(3):573-80). Further it was observed that IL-1a levels increase within the brain in post-stroke animal models during angiogenic periods (see, Salmeron K, et al., J Neurochem. 2016; 136(3):573-80). In the experiments described herein, IL-1a was significantly lower in HF-Saline treated mice compared to Control-Saline treated mice, and that treatment of P50, or A500 increases IL-1a significantly in comparison to HF-Saline mice. Suggesting that treatment with Ang-(1-7)/MasR agonists may provide brain protection and neurorepair by increasing brain IL-1α levels.

IL-2 has also been shown to play a protective role in the brain. IL-2 has been shown to decrease amyloid plaque load, improve synaptic plasticity, and has been associated with memory recovery in AD mice (see, Alves S, et al., Brain. 2017; 140(3):826-42). We (the inventors) observed that IL-2 brain levels significantly decreased in our VCID model in comparison to controls. We further demonstrated that treatment with P50, or A500 significantly increased IL-2 levels in comparison to HF-Saline mice, and that IL-2 levels were significantly negatively correlated with NfL levels. These data suggest that one of the mechanisms by which Ang-(1-7)/MasR treatment protects cognitive function may include increasing IL-2.

IL-5 is a Th2 cytokine that can act as an immunosuppressor (see, Schmitz M, et al., Neurobiol Aging. 2015; 36(9):2597-606) and contributes to maintaining homogeneity of astrocyte activation states (see, Zhou Y, et al., J Physiol Biochem. 2017; 73(2):259-66). Elevated serum levels of IL-5 have been have been reported in both VCID patients and vascular encephalopathy (VE) (see, Schmitz M, et al., Neurobiol Aging. 2015; 36(9):2597-606). It has been suggested that elevated levels of IL-5 in VCID and VE may contribute to disease reduction. In PC12 cells, treatments of IL-5 decreased neuronal cell apoptosis, and A025-35 induced tau phosphorylation (see, Zhou Y, et al., J Physiol Biochem. 2017; 73(2):259-66). Experiments described herein demonstrated that treatments with P50, or A50 significantly increase brain IL-5 levels compared to HF-Saline mice. These data suggest that elevated IL-5 levels may contribute to the mechanism by which Ang-(1-7)/MasR treatment protects cognitive function.

IL-13 has been shown to play both a protective and injurious role in the brain. While IL-13 has been demonstrated to be neuroprotective by modulating microglia/macrophage responses in traumatic brain injury (see, Miao W, et al., J Immunol. 2020; 204(6):1486-98), other studies have shown that IL-13 can be detrimental to neuronal survival (see, Morrison B E, et al., J Immunol. 2012; 189(12):5498-502). Experiments described herein found that treatment with P50, A50, and A500 significantly increase IL-13 levels in comparison to HF-Saline treated mice. The IL-13 levels were significantly negatively correlated to NfL plasma concentrations. These data indicate that IL-13 may play a neuroprotective role following treatment with Ang-(1-7)/MasR agonists in our mouse model of VCID.

IL-17 has also been shown to play both a protective and injurious role in the brain. Although IL-17 is typically linked to proinflammatory disease progression (see, Cipollini V, Anrather J, Orzi F, Iadecola C. Th17 and cognitive impairment: possible mechanisms of action. Front Neuroanat. 2019), it has also been shown to promote tissue repair via meningeal resident gamma delta T-cells and support long-term potentiation (LTP) and the plasticity of glutamatergic synapses during short-term learning (see, Bordon Y. IL-17: an immune mnemonic. Nat Rev Immunol. 2019). We observed that HF-Saline treated mice had significantly lower levels of IL-17 than Control-Saline treated mice, and that treatments with P50, A50, or A500 normalized IL-17 brain concentrations. Further, levels of IL-17 were significantly negatively correlated with Nfl levels. These findings suggest that Ang-(1-7)/MasR agonists increase IL-17 levels in VCID mice, which may contribute to these peptides' cognitive protective effects.

IP-10 (CXCL-10) has been demonstrated to increase in the pathogenesis of AD (see, McKimmie C, Michlmayr D. Role of CXCL10 in central nervous system inflammation. Int J Interf Cytokine Mediat Res. 2014; 6(1):1)[. In contrast, studies with AD patients demonstrate an increased in CSF and plasma IP-10 levels were independently associated with NfL levels (see, Bettcher B M, et al., J Alzheimers Dis. 2018; 62(1):385-97). In our model of VCID, we observed IP-10 brain levels were significantly higher in both Control-Saline-treated mice and Ang-(1-7)/MasR agonist-treated HF mice as compared to HF-Saline mice.

In total, the experiments described herein indicate that in the VCID model there is a decrease in the levels of multiple pleiotropic cytokines in HF-saline-treated animals. Treatment with Ang-(1-7)/MasR agonists mitigated cognitive impairment in this model while simultaneously increasing the levels of these pleotropic, and some putative neuroprotective cytokines, some of which are correlated to decreases blood NfL levels.

### Example 5

This example demonstrates increases in circulating neurodegeneration biomarkers neurofilament light protein and p-tau (e.g., p-tau181) in individuals with preserved cardiac function heart failure.

Systemic inflammation, which is present in heart failure (HF), is known to increase risk for dementia and development of vascular cognitive impairment (VCI). Experiments were conducted to determine if blood levels of neurofilament light (NfL) protein and tau phosphorylated at threonine-181 (p-tau181) are increased in HF patients at risk for VCI. This was a retrospective study of 58 participants from the previously completed “TOPCAT” trial (Treatment of Preserved Cardiac Function Heart Failure with an Aldosterone Antagonist). TOPCAT participants had symptomatic CHF, were >50 years of age, and had ejection fraction (LVEF)≥45%, assessed within 6 months from study start (n=58, 52% female). There were 58 participants, 25 subjects who were NYHA 1/2 at both baseline and month 12, 19 were NYHA 3/4 at both baseline and month 12, 6 subjects were NYHA 1/2 at baseline and then NYHA 3/4 at month 12, and 8 subjects were NYHA 3/4 at baseline and then NYHA 1/2 at month 12 (see, Table 5). Control serum samples were from age-matched adults between 50-85 with no known health issues (n=24, 50% female). NfL was measured using Simoa Quanterix and p-Tau181 measured with Simoa Human pTau-181 V2 kit. Samples were run and analyzed in duplicate. Statistical analysis was performed using GraphPad and significance tested using Welch's ANOVA, Dunnett's post. Baseline NfL levels were significantly higher in NYHA class I/II (n=56 samples, p=0.02) and NYHA class III/IV participants (n=54 samples, p<0.0001)) than in age-matched controls (see table for values) (Further, NfL levels in class III/IV were significantly higher than those seen with class I/II (ANOVA, Dunnett's post, p<0.001) suggesting that NfL levels were associated with HF severity. Over 12-months follow-up, participants in NYHA class I/II had an increase in NfL of 9.6±5% relative to baseline and those in class III/IV had a 31.7±17% increase from baseline. P-tau181 levels were also significantly higher in both NYHA class I/II (n=56 samples, p<0.0001) and III/IV (n=54 samples, p=0.0002) as compared to age-matched controls (see table for values) with levels were significantly higher in class III/IV than in class I/II participants (ANOVA, Dunnett's post, p<0.001). Levels of p-tau181 did not show significant changes over the 12-month time in any group.

These results demonstrated that 2 well-validated biomarkers of neurodegeneration, NfL and p-tau181, are elevated in subjects with HFpEF.

TABLE 5

| Median, (25, | | | |
|---|---|---|---|
| 75 Quartile) | | | |
| *, p = .027 | Age- | | |
| **, p < .0001 | matched | | BaselineNYHA |
| #, p < .0001 | controls | BaselineNYHA | Class III/IV |
| ##, p = .0002 | (n = 43 | Class I/II (n = | (n = 54 |
| Compared to | samples, | 56 samples, 31 | samples, 27 |
| control | 24 subjects) | subjects) | subjects) |
| NfL serum | 11.4 (7.0, | 16.9 (12.6, | 26.0 (16.1, |
| levels (pg/ml) | 16.6) | 22) * | 38.5) ** |
| p-tau181 serum | 0.19 (.07, | 0.54 (0.25, | 1.05 (0.45, |
| levels (pg/ml) | 0.3) | 1.6) # | 2.36) ## |

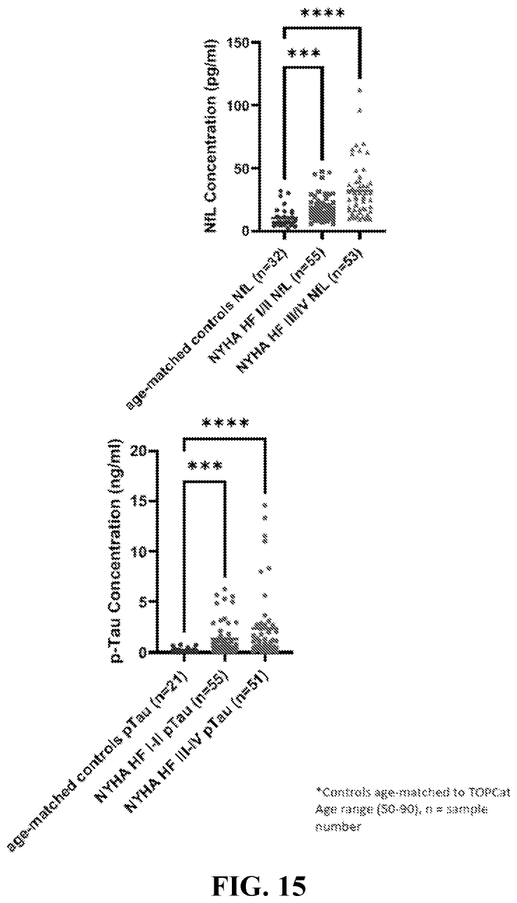

FIG. 15 shows that serum NfL and pTau increased with severity of heart failure.

The foregoing discussion of the invention has been presented for purposes of illustration and description. The foregoing is not intended to limit the invention to the form or forms disclosed herein. Although the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter. All references cited herein are incorporated by reference in their entirety.

1-2. (canceled)

3. A method for treating a subject diagnosed as having cognitive impairment comprising:
(a) determining from a blood fraction a neurofilament light protein (NFL) concentration and/or p-tau181 concentration in the subject, wherein the blood fraction is selected from the group consisting of serum, plasma, and whole blood;
(b) comparing the NFL level measured in step (a) and/or the p-tau181 level measured in step (a) to a pre-determined reference level; and
(c) administering a therapeutically agent to the subject if the NFL level measured in step (a) and/or the p-tau181 level measured in step (a) is greater than the reference level, wherein the therapeutic agent is a Mas receptor agonist and/or a p-tau181 inhibitor;
wherein the cognitive impairment is selected from the group consisting of vascular cognitive impairment, post-surgical cognitive impairment, and Alzheimer's disease.

4-7. (canceled)

8. The method of claim 3, wherein the Mas receptor agonist consists of an amino acid sequence of any one of SEQ ID NOs: 2-27, wherein the Mas receptor agonist is glycosylated.

9. (canceled)

10. The method of claim 3, wherein the Mas receptor agonist is an oligopeptide having the formula: A¹-A²-A³-A⁴-A⁵-A⁶-A⁷-A⁸ (SEQ ID NO:1) wherein
A¹ is selected from the group consisting of aspartic acid, glutamic acid, alanine, and glycosylated forms thereof;
A² is selected from the group consisting of arginine, histidine, lysine, and glycosylated forms thereof;
A³ is selected from the group consisting of valine, alanine, isoleucine, leucine, and glycosylated forms thereof;
A⁴ is selected from the group consisting of tyrosine, phenylalanine, tryptophan, and glycosylated forms thereof;
A⁵ is selected from the group consisting of isoleucine, valine, alanine, leucine, and glycosylated forms thereof;
A⁶ is selected from the group consisting of histidine, arginine, lysine, and glycosylated forms thereof;
A⁷ is selected from the group consisting of proline, glycine, serine, and glycosylated forms thereof; and
A⁸ can be present or absent, wherein when A⁸ is present, A⁸ is selected from the group consisting of serine, threonine, hydroxyproline, and glycosylated forms thereof,
wherein at least one of A¹-A⁸ is glycosylated with a monosaccharide or disaccharide, wherein at least one of the monosaccharides or disaccharides is selected from the group consisting of glucose, galactose, xylose, fucose, rhamnose, lactose, cellobiose, and melibiose.

11. The method of claim 10, wherein (a) A⁷ is terminated with an amino group and A⁸ is absent or (b) A⁸ is terminated with an amino group.

12-14. (canceled)

15. The method of claim 10, wherein A⁸ is glycosylated with a monosaccharide or disaccharide or A⁸ is absent and A⁷ is glycosylated with a monosaccharide or disaccharide, wherein at least one of the monosaccharides or disaccharides is selected from the group consisting of glucose, galactose, xylose, fucose, rhamnose, lactose, cellobiose, and melibiose.

16. (canceled)

17. The method of claim 15, wherein (a) A⁷ is terminated with an amino group and A⁸ is absent or (b) A8 is terminated with an amino group.

18. The method of claim 3, wherein (a) A⁷ is a serine or a glycosylated form thereof and A⁸ is absent or (b) A⁸ is serine or a glycosylated form thereof.

19. The method of claim 18, wherein (a) A⁷ is glycosylated with glucose or lactose and A⁸ is absent or (b) A⁸ is glycosylated with glucose or lactose.

20. The method of claim 18, wherein (a) A⁷ is terminated with an amino group and A⁸ is absent or (b) A⁸ is terminated with an amino group.

21. The method of claim 10, wherein the oligopeptide is PN-A5.

22. The method of claim 10, wherein the oligopeptide is PN-A6.

23. The method of claim 10, wherein the oligopeptide comprises at least one D-amino acid.

24. A method for diagnosing cognitive impairment in a human subject comprising:
(a) determining from a blood fraction a neurofilament light protein (NFL) and/or p-Tau181 concentration in the subject;
(b) comparing the NFL level and/or p-tau181 level measured in step (a) to a pre-determined reference level; and
(c) diagnosing the subject as having cognitive impairment when the NLF level measured in step (a) and/or the p-tau181 level measured in step (a) exceed the reference level.

25. The method of claim 24, wherein the cognitive impairment is selected from the group consisting of vascular cognitive impairment, post-surgical cognitive impairment, and Alzheimer's disease.

26. The method of claim 24, wherein the reference level is about 25 pg/ml in serum.

27. The method of claim 24, wherein the reference level is about 75 pg/ml in serum.

28-55. (canceled)

56. The method of claim 3, wherein the reference level is about 25 pg/ml in serum.

57. The method of claim 3, wherein the reference level is about 75 pg/ml in serum.
